(19) United States
(12) Patent Application Publication
(10) Pub. No.: US 2009/0117108 A1
(43) Pub. Date: May 7, 2009

(54) Gene Engineering Recombinant Anti-CEA, Anti-CD3, And Anti-CD28 Single-Chain Tri-Specific Antibody

(76) Inventors: Xiangbin Wang, Beijing (CN); Hualiang Huang, Beijing (CN); Baofeng Zhao, Beijing (CN); Qi Zhao, Beijing (CN); Jinhua Piao, Beijing (CN); Qing Lin, Beijing (CN)

Correspondence Address: JONES DAY, 222 EAST 41ST ST, NEW YORK, NY, 10017, US

(21) Appl. No.: 10/594,908

(22) PCT Filed: Mar. 29, 2005
(86) PCT No.: PCT/CN2005/000408

§ 371 Date: Jan. 8, 2009

(30) Foreign Application Priority Data
Apr. 1, 2004 (CN) 200410032158.3

(51) Int. Cl. A61K 39/395; C07K 16/18; C12N 15/11; C12N 15/00; C12N 1/21; C12P 21/04

(52) U.S. Cl. 424/135.1; 530/387.3; 536/23.53; 435/320.1; 435/252.33; 435/69.6

(57) ABSTRACT

The invention is related to a recombinant single-chain tri-specific antibody made from anti-Tumor Associated Antigen (TAA) antibody, FC interlinker, anti-CD3 antibody, HSA interlinker and anti-CD28 antibody in turn. Particularly, the invention relates to an anti-CEA, anti-CD3, anti-CD28 recombinant single-chain tri-specific antibody, CEA-scTsAb, which was constructed with three tandem antibody fragments (anti-CEA scFv, anti-CD3 scFv and anti-CD28 single-domain antibody) linked by two interlinkers (FC interlinker, HSA interlinker), and could be appended by C myc tag or histidine tag ((His)₆-tag) at the C terminal. It also concerns a method for construction, expression and purification of the antibody. It also offers the encoded DNA sequence of the antibody, expression vectors and host cells for the vectors.

### 1. FIELD

This invention refers to the field of recombinant antibody, more concretely, refers to a recombinant anti-CEA/CD3/CD28 single-chain tri-specific antibody (scTsAb); The method for constructing, expressing and purifying the scTsAb; the vectors and Escherichia coli host cells containing the scTsAb.

### 2. BACKGROUND

The activation of T lymphocytes needs two kinds of signals in vivo: the interaction between MHC/antigen peptide complex on APC (antigen presenting cells) and TCR/CD3 complex on T lymphocytes provides the first signal; the interaction between the co-stimulatory receptor on APC and co-stimulatory molecule on T lymphocytes provides the second signal, that is co-stimulatory signal. It was accepted generally that T lymphocytes cannot be activated fully only in the presence of the first signal (Baxter and Hodgkin, 2002; Bernard et al., 2002)

There are two kinds of T lymphocytes: cytotoxic T lymphocytes (CTL) and T helper cells (TH). CTL is the major effector cell in cellular immunological responses, while TH participate in cellular immunological responses indirectly by secreting cytokines (such interleukin-2 (IL-2)). As tumor immunity majors in cellular immunity, designing anti-tumor drugs to activate CTL specifically is of great importance in tumor immunotherapy. (Foss, 2002)

Now, a series of recombinant anti-tumor/CD3 bispecific antibodies (BsAbs) have been designed to provide the first signal for CTL activation, among which some have entered into clinic research (Daniel et al., 1998; Holliger et al., 1999; Loffler et al., 2000; Manzke et al., 2001a; Manzke et al., 2001b; Dreier et al., 2002; Dreier et al., 2003; Loffler et al., 2003; Min Fang, 2003; Fang et al., 2004). In summary of previous results, BsAbs had been proved to activate T lymphocytes specifically and induce tumor specific cytolysis obviously. However, providing no co-stimulatory signal, most of them could not activate T lymphocytes fully and may result in activation induced cell death (AICD) of T lymphocytes (Daniel et al., 1998), and reduce their tumor specific cytolysis (Daniel et al., 1998).

To overcome above defects, another kind of BsAb was designed: anti-tumor/CD28 BsAb. In company with anti-tumor/CD3 BsAb, they provides CTLs with dual activating signals and induces more efficient tumor specific cytolysis (Jung et al., 2001; Kodama et al., 2002). However, there are several disadvantages for combinatorial using of above two BsAbs, such as the duplicate steps in expression and purification, the consequential increase of production cost, and the partnership of two BsAb in clinical medication. Tri-specific antibody (TsAb) with three binding specificities (to TAA, CD3 and CD28) may replace above two BsAbs in providing dual activating signals in a single molecule and be superior to them in expression, purification and clinical medication.

Heretofore, there are three types of scTsAb: chemical conjugating TsAb (Jung et al., 1991; Tutt et al., 1991; French, 1998; Wong et al., 2000), recombinant polymeric TsAb (Atwell et al., 1999; Dolezal et al., 2000; Schoonjans et al., 2000a; Schoonjans et al., 2000b; Kortt et al., 2001; Schoonjans et al., 2001; Willems et al., 2003) and the type of recombinant single-chain tri-specific antibody (scTsAb)(Li-ping et al., 2003; Zhang et al., 2003). The third type of TsAb is believed to be superior to others for its simplification in construction, expression and purification. Also, as carcinoembryonic antigen (CEA) is a broad-spectrum TAA (Shi et al., 1983; Ganjei et al., 1988; Horie et al., 1996; Kuo et al., 1996; Feil et al., 1999; Tomita et al., 2000; Kammerer et al., 2003), scTsAb containing anti-CEA antibody may be used in preventing or treating diverse tumors in clinic.

### SUMMARY OF INVENTION

The introduction of anti-CEA antibody in anti-CEA/CD3/CD28 scTsAb in this invention provides a convenience to distinguish tumor cells from normal cells in vivo, and avoid or decrease the non-specific killing by activated T lymphocytes.

In another aspect of this invention, as CEA is widely expressed on many tumor cells, it also provides a broad application for treating or preventing different tumor in future. The invention provides an anti-CEA/CD3/CD28 scTsAb for treating or preventing different tumor.

In another aspect of this invention, it provides a method for constructing scTsAb.

The amino acid sequence (SEQ ID NO: 1) of murine anti-CEA single chain fragment of variable region contained in CEA-scTsAb is listed:

QVQLQQSGAELMKPGASVKISCKATGYTFSDYWIEWVKQRPGHGLEWIGE
ILPGSGRTDYNERFKGKATFTGDVSSNTAYMKLSSLTSEDSAVYYCATGT
TPFGYWGQGTLVTVSATSTPSHNSHQVPSAGGPTANSGSRDIVLTQSPAS
LAVSLGQRATISCRASQSVSTSSYTYMHWYQQKPGQPPKLLIKYASNLES
GVPARFSGSGSGTDFTLNIHPVEEEDTAYYYCQHSWEIPRTFGGGTKLEI
K

The amino acid sequence (SEQ ID NO: 2) of anti-CD3 single chain fragment of variable region contained in CEA-scTsAb is listed:

EVKLVESGPELVKPGASMKISCKASGYSFTGYTMNWVKQSHGKNLEWMGL
INPYKGVSTYNQKFKDKATLTVDKSSSTAYMELLSLTSEDSAVYYCARSG
YYGDSDWYFDVWGAGTSVTVSSTSGGGGSGGGGSGGGGSSRDIQMTQTTS
SLSASLGDRVTISCRASQDIRNYLNWYQQKPDGTVKLLIYYTSRLHSGVP
SKFSGSGSGTDYSLTISNLEQEDIATYFCQQGNTLPWTFAGGTKLELKRA

The nucleic acid sequence (SEQ ID NO: 3) of CEA-scTsAb is listed:

| | |
|---|---|
| 1 | ATGGGTCTCGAGCAGGTGCAGCTGCAGCAGAGCGGTGCGGAACTGATGAA |
| 51 | ACCGGGCGCGAGCGTGAAAATCAGCTGCAAAGCGACCGGCTATACCTTCA |
| 101 | GCGATTATTGGATCGAATGGGTGAAACAGCGTCCGGGTCACGGCCTGGAA |
| 151 | TGGATCGGTGAAATCCTGCCGGGCAGCGGCCGTACCGACTACAACGAACG |
| 201 | TTTCAAAGGCAAAGCGACCTTCACCGGCGACGTTTCTAGCAACACCGCGT |
| 251 | ATATGAAACTGTCTAGCCTGACCAGCGAAGATAGCGCGGTGTATTACTGC |
| 301 | GCGACCGGCACCACCCCGTTCGGTTACTGGGGTCAGGGCACCCTGGTTAC |
| 351 | CGTTTCCGCGACTAGTACCCCGAGCCATAACAGCCATCAGGTGCCGAGCG |
| 401 | CGGGCGGCCCGACCGCGAACAGCGGCTCTAGAGACATCGTGCTGACCCAG |
| 451 | AGCCCGGCGAGCCTGGCGGTGTCTCTGGGTCAGCGTGCGACCATCTCCTG |
| 501 | CCGTGCTTCCCAGTCCGTTTCCACCTCCTCCTACACCTACATGCACTGGT |
| 551 | ATCAGCAGAAACCGGGTCAGCCGCCGAAACTGCTGATCAAATATGCGAGC |
| 601 | AACCTGGAATCTGGTGTGCCGGCGCGTTTCAGCGGTTCTGGCAGCGGCAC |
| 651 | CGACTTCACCCTGAACATCCACCCGGTGGAAGAAGAAGATACCGCGTATT |
| 701 | ACTATTGCCAGCACTCTTGGGAAATCCCGCGTACCTTCGGTGGCGGCACC |
| 751 | AAACTGGAAATCAAAGAATTCAACAGCACGTACCGGGTTGTAAGCGTCCT |
| 801 | CACCGTACTGCACCAGGACTGGCTGAATGGCAAGGAATACAAATGCAAGA |
| 851 | GTACTGAGGTGAAGCTGGTGGAGTCTGGACCTGAGCTGGTGAAGCCTGGA |
| 901 | GCTTCAATGAAGATATCCTGCAAGGCTTCTGGTTACTCATTCACTGGCTA |
| 951 | CACCATGAACTGGGTGAAGCAGAGTCATGGAAAGAACCTTGAGTGGATGG |
| 1001 | GACTTATTAATCCTTACAAAGGTGTTAGTACCTACAACCAGAAGTTCAAG |
| 1051 | GACAAGGCCACATTAACTGTAGACAAGTCATCCAGCACAGCCTACATGGA |
| 1101 | ACTCCTCAGTCTGACATCTGAGGACTCTGCAGTCTATTACTGTGCAAGAT |
| 1151 | CGGGGTACTACGGTGATAGTGACTGGTACTTCGATGTCTGGGGCGCAGGA |
| 1201 | ACCTCAGTCACTGTCTCCTCAACTAGTGGTGGTGGTGGTTCTGGTGGTGG |
| 1251 | TGGTTCTGGTGGTGGTGGTTCTTCTAGAGACATCCAGATGACCCAGACCA |
| 1301 | CATCCTCCCTGTCTGCCTCTCTGGGAGACAGAGTCACCATCAGTTGCAGG |
| 1351 | GCAAGTCAGGACATTAGAAATTATTTAAACTGGTATCAACAGAAACCAGA |
| 1401 | TGGAACTGTTAAACTCCTGATCTACTACACATCAAGATTACACTCAGGAG |
| 1451 | TCCCATCAAAGTTCAGTGGCAGTGGGTCTGGAACAGATTATTCTCTCACC |
| 1501 | ATTAGCAACCTGGAGCAAGAGGATATTGCCACTTACTTTTGCCAACAGGG |
| 1551 | TAATACGCTTCCGTGGACGTTCGCTGGAGGCACCAAACTGGAACTGAAGC |
| 1601 | GCGCTGTCGACTTCCAGAATGCGCTGCTGGTTCGTTACACCAAGAAAGTA |
| 1651 | CCCCAAGTGTCAACTCCAACTCCTGTAGAGGTCTCACATATGCAGGTACA |
| 1701 | GCTACAGGAATCTGGTCCGGGTCTGGTAAAACCGTCTCAGACCCTGTCTC |
| 1751 | TGACCTGTACCGTATCTGGTTTCTCTCTGTCTGACTATGGTGTTCATTGG |
| 1801 | GTACGTCAGCCGCCAGGTAAAGGTCTGGAATGTCTGGGTGTAATATGGGC |
| 1851 | TGGTGGAGGCACGAATTATAATTCGGCTCTCATGTCCAGACGTGTAACCT |
| 1901 | CTTCCGACGATACCTCTAAAAATCAGTTCTCTCTGAAACTGTCTCTGTCT |
| 1951 | TCCGTAGACACCGCTGTATACTATTGTGCTCGTGACAAAGGTTACTCCTA |
| 2001 | TTACTATTCTATGGACTACTGGGGTCAGGGCACCCTGGTAACCGTATCTT |
| 2051 | CCGGTACCGAACAAAAACTCATCTCAGAAGAGGATCTGAATGGGGCCGCA |
| 2101 | CATCATCATCACCATCACGAGCAA |

The amino acid sequence (SEQ ID NO: 4) of CEA-scTsAb is listed:

MGLEQVQLQQSGAELMKPGASVKISCKATGYTFSDYWIEWVKQRPGHGLE
WIGEILPGSGRTDYNERFKGKATFTGDVSSNTAYMKLSSLTSEDSAVYYC
ATGTTPFGYWGQGTLVTVSATSTPSHNSHQVPSAGGPTANSGSRDIVLTQ
SPASLAVSLGQRATISCRASQSVSTSSYTYMHWYQQKPGQPPKLLIKYAS
NLESGVPARFSGSGSGTDFTLNIHPVEEEDTAYYYCQHSWEIPRTFGGGT
KLEIKEFNSTYRVVSVLTVLHQDWLNGKEYKCKSTEVKLVESGPELVKPG
ASMKISCKASGYSFTGYTMNWVKQSHGKNLEWMGLINPYKGVSTYNQKFK
DKATLTVDKSSSTAYMELLSLTSEDSAVYYCARSGYYGDSDWYFDVWGAG
TSVTVSSTSGGGGSGGGGSGGGGSSRDIQMTQTTSSLSASLGDRVTISCR
ASQDIRNYLNWYQQKPDGTVKLLIYYTSRLHSGVPSKFSGSGSGTDYSLT
ISNLEQEDIATYFCQQGNTLPWTFAGGTKLELKRAVDFQNALLVRYTKKV
PQVSTPTPVEVSHMQVQLQESGPGLVKPSQTLSLTCTVSGFSLSDYGVHW
VRQPPGKGLECLGVIWAGGGTNYNSALMSRRVTSSDDTSKNQFSLKLSLS
SVDTAVYYCARDKGYSYYYSMDYWGQGTLVTVSSGTEQKLISEEDLNGAA
HHHHHHEQ

In another aspect of this invention, it provides a vector for expressing CEA-scTsAb: CEA-scTsAb/pTRI.

In another aspect of this invention, it provides an Escherichia coli host cell containing above vector.

In another aspect of this invention, it provides a method for promoting cytoplasmic soluble expression of above scTsAbs at lower temperature.

In another aspect of this invention, it provides a method for purifying above scTsAbs with DEAE anion exchange chromatography.

However, in the context of this invention, other aspects and advantages of this invention are obvious to the ordinary persons engaged in the similar field, especially based on that disclosed in “example” part.

### BRIEF DESCRIPTION OF DRAWINGS

FIG. 1. The diagram process for constructing multi-cloning DNA frame with overlapping PCR. The numbers from 2 to 11 represent different synthetic fragments of polymeric nucleic acid. The signs of “A, B, C, D, E, I, II, III, IV, UP, DOWN” represent the semi-finished products of construction. The sign of “WHOLE” represents the ultimate product.

FIG. 2. Detection of the over-lapping PCR product by Agarose Gel electrophoresis. Lane 1: the product of over-lapping PCR; Lane 2: DL2000 DNA marker (Dalian Takara Biotech.).

FIG. 3. The sequence, restriction sites and constitution of multi-cloning DNA fragment.

FIG. 4. The process for constructing CEA-scTsAb.

FIG. 5. The diagram maps of parent and ultimate vectors for constructing and expressing CEA-scTsAb.

FIG. 6. Identification of the constructing process by Agarose Gel electrophoresis. Lane 1: PCR product amplified from empty vector pTRI; Lane 2: PCR product amplified from vector CD28 VH/pTRI; Lane 3: PCR product amplified from vector CD3scFv/CD28 VH/pTRI; Lane 4: PCR product amplified from vector CEA-scTsAb/pTRI; Lane 5: DL2000 DNA marker (Dalian Takara Biotech.).

FIG. 7. The diagram process for constructing murine anti-CEA scFv with overlapping PCR. The numbers from 1 to 22 represent different synthetic fragments of polymeric nucleic acid. The signs of “A, B, C, D, E, F, G, H, I, J, K, a, b, c, d, e, f, g, I, II, III, IV, UP, DOWN” represent the semi-finished products of construction. The sign of “WHOLE” represents the ultimate product.

FIG. 8. Detection of the over-lapping PCR product by Agarose Gel electrophoresis. Lane 1 and 9: DL2000 DNA marker (Dalian Takara Biotech.). Lane 2-5: semi-finished products I, II, III, IV; Lane 6 and lane 7: semi-finished products UP and DOWN; Lane 8: the ultimate product WHOLE.

FIG. 9. SDS-PAGE of soluble expression of CEA-scTsAb. Lane 1: the ultrasonic deposition of CEA-scTsAb/pTRI expression; Lane 2: the ultrasonic supernatant of CEA-scTsAb/pTRI expression; Lane 3: the protein molecular weight standard (Shanghai Biochemistry Institute); Lane 4: the ultrasonic deposition of empty vector pTRI expression; Lane 5: the ultrasonic supernatant of empty vector pTRI expression. The bands of CEA-scTsAb are arrowed in corresponding lanes.

FIG. 10. Western-blotting of soluble expression of CEA-scTsAb. Lane 1: the protein molecular weight standard (NEB); Lane 2: the ultrasonic deposition of CEA-scTsAb/pTRI expression; Lane 3: the ultrasonic deposition of empty vector pTRI expression; Lane 4: the ultrasonic supernatant of CEA-scTsAb/pTRI expression; Lane 5: the ultrasonic supernatant of empty vector pTRI expression.

FIG. 11. SDS-PAGE of purification with DEAE anion exchange chromatography of CEA-scTsAb. Lane 1: the ultrasonic supernatant of empty vector pTRI expression; Lane 2: the ultrasonic supernatant of CEA-scTsAb/pTRI expression; Lane 3: the flow-through of DEAE anion exchange chromatography; Lane 4: the NaCl elution of DEAE anion exchange chromatography; Lane 5: the NaOH elution of DEAE anion exchange chromatography; Lane 6: the protein molecular weight standard (Shanghai Biochemistry Institute). The bands of CEA-scTsAb are arrowed in corresponding lanes.

FIG. 12. The ELISA (enzyme linked immunosorbent assay) result of CEA-scTsAb. From top to bottom, four cures represent four results. The first curve: 10 μg/ml Jurkate membrane antigen; the second one: 1 μg/ml purified CEA(R&D); the third one: 1 μg/ml CD28-FC chimera(R&D); the fourth one with no antigen coated.

FIG. 13. FACS of the binding of CEA-scTsAb to different tumor cells. The shadowed peak is the negative control with no CEA-scTsAb added; The blank one is the result added with CEA-scTsAb.

FIG. 14. FACS of the binding of CEA-scTsAb to Jurkate cells and peripheral blood mononuclear cells (PBMC). The shadowed peak is the negative control with no CEA-scTsAb; The blank one is the result added with CEA-scTsAb.

FIG. 15. MTT assay of the effect of E/T ratio (Effector cells/target cells) on tumor specific cytolysis induced by CEA-scTsAb. From top to bottom, three cures represent three different E/T. The first curve: E/T=10; the second one: E/T=5; the third one: E/T=1. Effector cells: PBMC. Target cells: SW1116 tumor cells.

FIG. 16. MTT assay of the effect of CEA-scTsAb concentration on tumor specific cytolysis. There are four stepwise phases for tumor specific cytolysis. In the first phase from 6 μg/ml to 12 μg/ml, the efficiency of tumor specific cytolysis displays negative correlation with the concentration of CEA-scTsAb and reaches the peak at 6 μg/ml; In the second phase from 750 ng/ml to 6 μg/ml, it displayes a direct correlation and reaches the bottom at 750 ng/ml; In the third phase from 24 ng/ml to 750 ng/ml, it turns back into negative correlation; In the fourth phase from 24 ng/ml to zero, the direct correlation appeared again.

FIG. 17. MTT assay of the effect of CEA-scTsAb concentration on the proliferation of effector cells. There are three stepwise phases for stimulating index (SI). In the first phase from 1.5 μg/ml to 12 μg/ml, SI displays direct correlation with the concentration of CEA-scTsAb and reaches the bottom at 1.5 μg/ml; In the second phase, it displays a negative correlation and reaches the peak at 47 ng/ml; In the third phase from 47 ng/ml to zero, it turns back into direct correlation and reaches the bottom at 0.7 ng/ml.

FIG. 18. The observation of the morphological changes of mixed cells in the process of tumor specific cytolysis induced by CEA-scTsAb. (A) SW116 tumor cells after 20 hours culture. (B)—(I) The mixture of SW1116 (target cells) and PBMC (effector cells) added with CEA-scTsAb (1 μg/ml) after 20 hours culture. E/T=5. (B) The adherent target cells begin to detach. (C) The effector cells aggregate on the surface of the target cells. (D) Stabs appeared on the surface of the target cells. (E) Partial membrane of target cells breaks up. (F) The whole membrane of target cells break up. (G) to (I) The target cells break into fragments.

FIG. 19. The mechanism diagram of tumor specific cytolysis induced by CEA-scTsAb. The upper map: the structure of CEA-scTsAb; the lower map: The mechanism diagram of tumor specific cytolysis induced by CEA-scTsAb: while CEA-scTsAb binding to target cell and effector cell simultaneously and activating effector cell by providing dual signals, target cell is killed specially.

FIG. 20. Fluorescence Photomicrography of killed target cells (SW1116) stained with PI and Annexin V-FITC (Fluorescein Isothiocyanate). Line A, B and C represents three different state of tumor cell death respectively: necrosis, late apoptosis and early apoptosis. The second column is the result of single green fluorescence; The third one is the result of single red fluorescence; The first one is the superposition of the other two image.

FIG. 21. FACS (PI/Annexin V-FITC of tumor specific cytolysis for killed tumor cells. Four quadrants represent different states of tumor cells: live cells in low left quadrant (LL); early apoptosis cells in low right quadrant (LR); late apoptosis cells in up right quadrant (UR); necrosis cells in up left quadrant (UL). The sample with no CEA-scTsAb added: LL(90.17%), LR(1.66%), UL(5.94%), UR(2.23%); The sample added with 50 ng/ml CEA-scTsAb: LL(52.83%), LR(16.12%), UL(9.8%), UR(21.25%).

### DETAILED DESCRIPTION

In this invention, all terms are easy to understand for ordinary workers engaged in this field except that is explained specially. Here, some terms are described as below:

Recombinant single-chain tri-specific antibody is the single linear antibody molecule constructed by genetic engineering method with three different antigen binding specificity. To speak concretely, recombinant anti-CEA/CD3/CD28single-chain tri-specific antibody is the single linear molecule constructed by fusing three different antibody fragments (anti-CEA antibody, anti-CD3 antibody, anti-CD28 antibody), interspaced with two inlinkers (FC linker and HSA linker)(Min Fang, 2003). As a alternative, C myc tag and histidine tag can be added at the C terminal of it for activity detection or further purification (Hengen, 1995; Fan et al., 1998) The antibody fragments mentioned here could be single chain fragment of variable region (scFv), Fab fragment of antibody or single domain antibody (VH or VL). More concretely, CEA-scTsAb is constructed by fusing anti-CEA scFv, FC interlinker, anti-CD3 scFv, HSA interlinker and anti-CD28 VH in tandem, with c-myc tag and histidine tag at its C terminal. The advantages for it are listed:
1. Basing on two signals-activating model for T cell, it is endowed with the ability of activating T cell fully.
2. As CEA is a broad-spectrum TAA, it is endowed with broad applications for treating or preventing many tumors in clinic.

The method for inducing cytoplasmic soluble expression of CEA-scTsAb at low temperature mentioned in this invention, requires that the host bacteria was induced with 0.4 mM IPTG at 30° C. to express CEA-scTsAb solubly in the cytoplasm. With this method, the ratio of inclusion body can be decreased remarkably, and about 50% of expressed CEA-scTsAb is soluble. The soluble expression of CEA-scTsAb can be used in further step of purification directly in no need of denaturation or renaturation, which will make for reducing the cost of production and improving the output.

The method of a single step of purification by collecting flow-through of DEAE anion exchange chromatography, require that the soluble expression product be loaded into the column filled with DEAE anion exchange resin at pH 8.0. Subsequently, almost all none-target proteins can be absorbed, while most of CEA-scTsAb flows through with about 75% purity.

The operating procedure of this invention is listed:

At first, the parent vector pTRI is constructed by introducing a new special Multi-Cloning Sites (MCS). Then the DNA fragment coding anti-CD28 VH is amplified with PCR from the vector, CD28 VH/pTMF, at both ends of which the special pair of restriction sites, Nde I/Kpn I, is added. With the same method, the DNA fragment coding anti-CD3 scFv with the restriction sites, ScaI/SalI, is prepared. The DNA fragment coding anti-CEA scFv with the restriction sites, XhoI/EcoRI, is cut from CEA scFv/pTMF. At last, all above three fragments are introduced into pTRI in tandem to produce the final vector CEA scTsAb/pTRI, in which the DNA fragments coding anti-CEA scFv, anti-CD3 scFv or anti-CD28 VH are arrayed in tandem from N end to C end.

Being transformed into E. coli BL21(DE3) and induced with IPTG at lower temperature (30° C.), CEA-scTsAb is expressed in cytoplasm solubly. With a further single step of DEAE anion exchange chromatography, it is purified primarily. The binding specificities to three antigens (CEA, CD3, CD28) are detected by ELISA; The binding specificities to tumor cells are detected by single color FACS after conjugating CEA-scTsAb with FITC; The cytolysis of tumor cells, the proliferation of T lymphocytes induced by CEA-scTsAb are both analyzed by MTT assay; The morphological changes of tumor cells are recorded by microphotography with inverted microscope. With dual-color FACS, PI/annexin-V-FITC, and fluorescence-microscope, the necrosis and apoptosis of tumor cells induced by CEA-scTsAb are visualized.

### EXAMPLES

This invention will be described in detail below referring to appended drawings. To be comprehended, all examples below are listed here to illuminate the invention, not to restrict it.

### Example 1

To prepare the DNA fragment containing multiple cloning sites by overlapping PCR.

The schematic process is shown in FIG. 1. All synthetic fragments used here are listed:

(SEQ ID NO:5)

| | |
|---|---|
| 1. | 5′-TAT ACC ATG GGT CTC GAG-3′ |

(SEQ ID NO:6)

| | |
|---|---|
| 2. | 5′-TAT ACC ATG GGT CTC GAG ATG TAC CCG CGC GGT |
| | AAC ACT AGT GAA TTC AAC AGC ACG TA-3′ |

(SEQ ID NO:7)

| | |
|---|---|
| 3. | 5′-AGC CAG TCC TGG TGC AGT ACG GTG AGG ACG CTT |
| | ACA ACC CGG TAC GTG CTG TTGAAT TC-3′ |

(SEQ ID NO:8)

| | |
|---|---|
| 4. | 5′-CTG CAC CAG GAC TGG CTG AAT GGC AAG GAA TAC |
| | AAA TGC AAG AGT ACT TCT AGA ATG TA-3′ |

(SEQ ID NO:9)

| | |
|---|---|
| 5. | 5′-CGA ACC AGC AGC GCA TTC TGG AAG TCG ACG TTA |
| | CCG CGC GGG TAC ATT CTA GAA GTA CT-3′ |

(SEQ ID NO:10)

| | |
|---|---|
| 6. | 5′-AAT GCG CTG CTG GTT CGT TAC ACC AAG AAA GTA |
| | CCC CAA GTG TCA ACT CCA ACT CCT GT-3′ |

(SEQ ID NO:11)

| | |
|---|---|
| 7. | 5′-GCG GTA CCG TTA CCG CGC GGG TAC ATC ATA TGT |
| | GAG ACC TCT ACA GGA GTT GGA GTT GA-3′ |

(SEQ ID NO:12)

| | |
|---|---|
| 8. | 5′-CGC GGT AAC GGT ACC GCG CTG GAA GTT GAC GAA |
| | ACC TAC GTT CCG AAA GAA TTT AAC GC-3′ |

(SEQ ID NO:13)

| | |
|---|---|
| 9. | 5′-TCG CTA GCC CCA TCC GCG GGA TGT CAG CGT GGA |
| | AGG TGA AGG TTT CCG CGT TAA ATT CTT TCG G-3′ |

(SEQ ID NO:14)

| | |
|---|---|
| 10. | 5′-ATC GAG CTC ATG TAC CCG CGC GGT AAC GCT AGC |
| | GAA CAA AAA CTC ATC TCA GAA GAG GA-3′ |

(SEQ ID NO:15)

| | |
|---|---|
| 11. | 5′-TA TTG CTC GTG ATG GTG ATG ATG ATG TGC GGC |
| | CCC ATT CAG ATC CTC TTC TGA GAT GAG-3′ |

(SEQ ID NO:16)

| | |
|---|---|
| 12. | 5′-CTC GAC GGA TCC TTA TTG CTC GTG ATG GTG-3′ |

The operating steps:

Step 1: according to FIG. 1, to mix the fragments (from 2 to 11) in pair and carry out the elongating reaction as below. All products are collected without any purification and applied in next step directly.

Reaction mixture: the synthetic fragments, 1 μl (each); 10×PCR buffer, 2 μl; dNTPs (2 mmol/ml each) (Dalian TaKaRa Biotechnology Co. Ltd.), 2111; Taq (1U) (Dalian TaKaRa Biotechnology Co. Ltd.) 0.5 μl; distilled water, 14 μl.

Reaction condition: to pre-denature at 94° C. for 1 minute; to denature at 94° C. for 30 seconds; to anneal at 45° C. for 30 seconds; to elongate at 72° C. for 30 seconds; 10 cycles.

Step 2: according to FIG. 1, to mix the products (A, B, C, D, E) of step 1 in pair and carry out the elongating reaction as below without any primers. All products are applied to agarose electrophoresis (1%) and purified by DNA Gel purifying Kit (Watson Biotech. Inc.). The product I (A with B) is about 180 bp; the product II (B with C) is about 180 bp; the product III (C with D) is about 180 bp; the product IV (D with E) is about 100 bp. Reaction mixture: the products of step 1, 10 μl (each). The reaction was carried out as below without any other components:

To pre-denature at 94° C. for 1 minute; to denature at 94° C. for 30 seconds; to anneal at 45° C. for 30 seconds; to elongate at 72° C. for 30 seconds; 10 cycles.

Step 3: according to FIG. 1, to mix the products (I, II, III, IV) of step 2 in pair and carry out the elongating reaction as below. All products are applied to agarose electrophoresis (1%) and purified by DNA Gel purifying Kit (Watson Biotech. Inc.). The product UP (I with II) is about 340 bp; the product DOWN (III with IV) is about 260 bp.

Reaction mixture: the products of step 2, 1 μl (each); 10× PCR buffer, 2 μl; dNTPs (2 mmol/ml each) (Dalian TaKaRa Biotechnology Co. Ltd.), 2 μl; Taq (1U) (Dalian TaKaRa Biotechnology Co. Ltd.) 0.5 μl; distilled water, 13 μl.

Reaction condition: to pre-denature at 94° C. for 1 minute; to denature at 94° C. for 30 seconds; to elongate at 72° C. for 50 seconds; 25 cycles.

Step 4: according to FIG. 1, to mix the product (UP, DOWN) of step 3 in pair and carry out the amplifying reaction with synthetic fragment 1 and 12 as primers:

Reaction mixture: the products of step 3 (UP, DOWN), 1 μl (each); primers (synthetic fragment 1 and 12), 1 μl; 10×PCR buffer, 2 μl; dNTPs (2 mmol/ml each) (Dalian TaKaRa Biotechnology Co. Ltd.), 2 μl; Taq (1U) (Dalian TaKaRa Biotechnology Co. Ltd.) 0.5 μl; distilled water, 12 μl.

Reaction condition: to pre-denature at 94° C. for 1 minute; to denature at 94° C. for 30 seconds; to elongate at 72° C. for 50 seconds; 25 cycles.

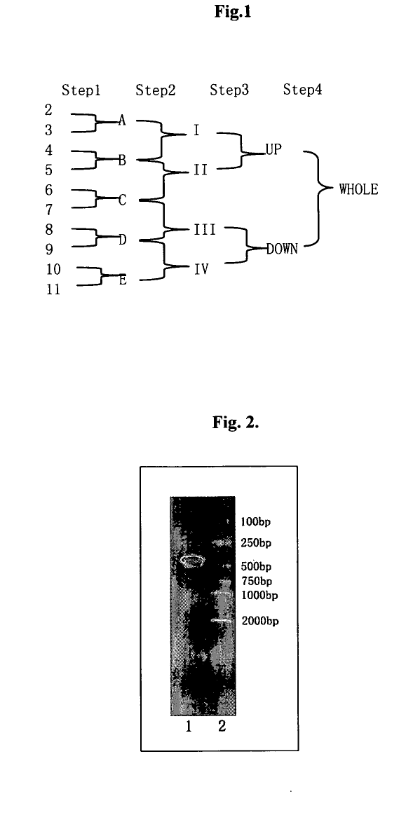

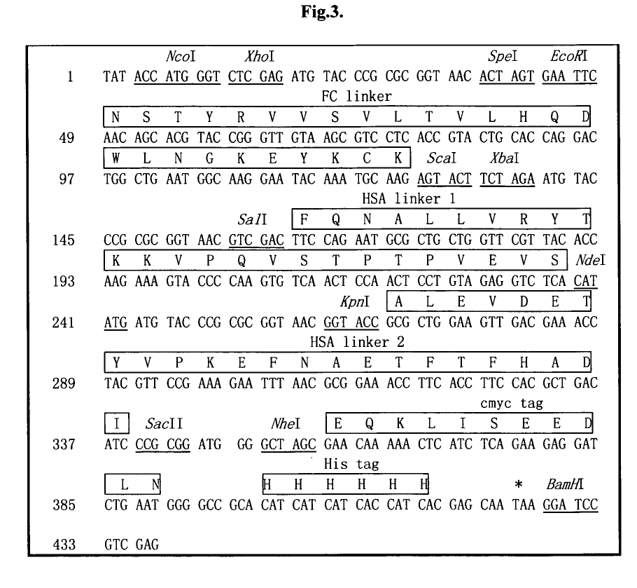

The ultimate products (439 bp) are applied to agarose electrophoresis (1%) (FIG. 2.) and purified by DNA Gel purifying Kit (Watson Biotech. Inc.). The sequence, restriction sites and its components are shown in FIG. 3.

### Example 2

### Construction of CEA-scTsAb

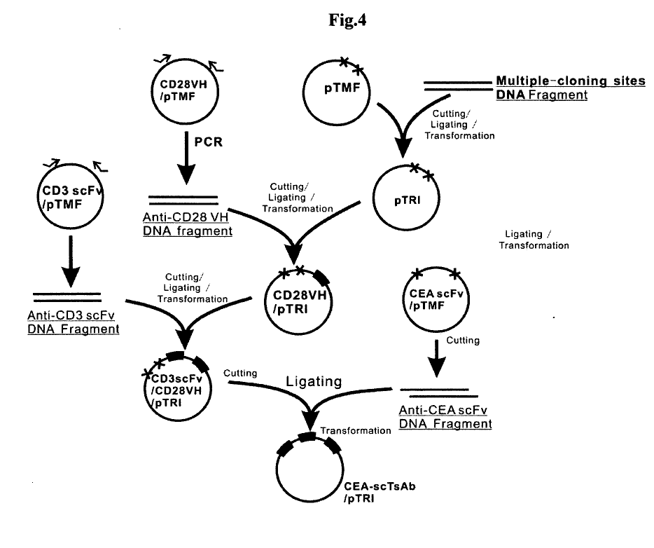

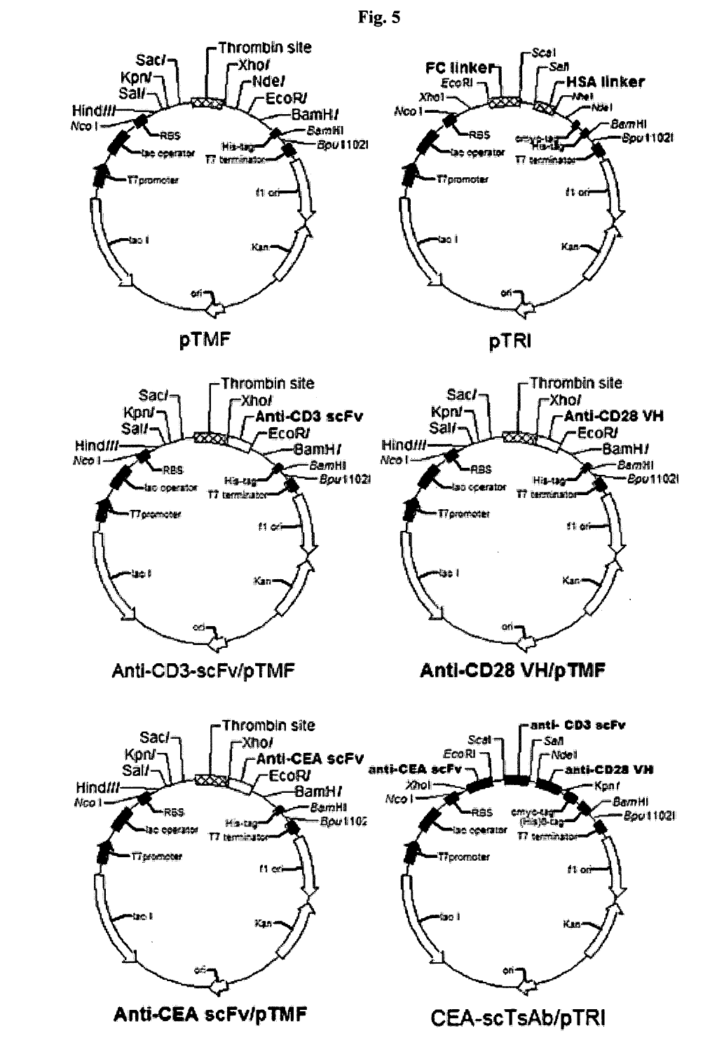

The diagram process of construction is shown in FIG. 4, and the schematic map of all vectors used in the process are listed in FIG. 5. The construction steps are listed:

(1) Construction of pTRI Vector

The DNA fragment containing multiple cloning sites and empty vector pTMF (Zhang et al., 2003) are both cut with NcoI/BarnHI and ligated together. The products of ligating are transformed into E. coli strain TOP10 (Invitrogen). The plasmid isolated from the transformed bacterial cells is named pTRI and used for next step.

Restriction enzyme digesting, ligating, preparation of TOP10 competent cells and transformation are carried out as below:

Restriction enzyme digesting reaction: in a volume of 20 μl, 1 μg of pTMF or the DNA fragment containing multiple cloning sites are digested with NcoI/BamHI (Promega) according to the operating manual. The products are applied to agarose electrophoresis (1%) and purified by DNA Gel purifying Kit (Watson Biotech. Inc.). The digested product for pTMF is about 5000 bp, while that of the DNA fragment containing multiple cloning sites if about 430 bp. Ligating reaction: 50-100 ng cut vector and 3-10 times (mol ratio) cut DNA fragments are mixed in a volume of 20 μl which contained 2 μl 10× T4 DNA ligase buffer, 1U T4 DNA ligase (Dalian TaKaRa Biotechnology Co. Ltd.) and necessary distilled water. The Ligating reaction is carried out at 16° C. overnight.

Preparation of Top10 Competent Cells: to Inoculate the Top10 Bacteria (Invitrogen Co.) to 2 ml LB medium ((10 g/l tryptone (GIBCO Co.), 5 g/l yeast extract (GIBCO Co.), 5 g/l NaCl, pH 7.5)), and incubate overnight at 37° C. with shaking. Then transfer to 20-40 ml LB medium at the rate of 1:100, incubate at 37° C. with shaking to reach A600 0.3-0.4 (about 2.5 hour). To chill on ice for 15 minutes and centrifuge at 4° C. at 4000 rpm for 10 minutes. The pellet is suspended in 10 ml of pre-chilled 0.1 mol/l CaCl₂ (Sigma Co.) and chilled on ice for 20 minutes. After the second centrifuge at 4° C. at 4000 rpm for 10 minutes, the pellet is gently suspended in 1˜2 ml of pre-chilled 0.1 mol/l CaCl₂ solution with 12% glycerol, and divided the aliquot of 200 μl in each EP tube, stored at −80° C.

Transformation: the ligating mixture is added into 200 μl competent cells. After being mixed gently and chilled on ice for 30 minutes, it is put in water bathe of 42° C. for 100 seconds, and then chilled on ice for 2 minutes. After adding 0.8 ml LB medium into the mixture, to shake it at 37° C. (<150 rpm) for 45 minutes to recover the cells. At last, the cells are centrifuged at 10,000 rpm for 1 minute, re-suspended in 50˜100 μl LB medium, spread onto the LB-K plate (10 g/l tryptone, 5 g/l yeast extract, 5 g/l NaCl, 15 g/l agar (SIGMA Co.), 50 μg/ml kanamycin (SIGMA Co.), pH 7.5) and incubated at 37° C. overnight.

Selection of positive clones: to pick the single clones on the LB-K plate and transfer them into 2 ml LB-K medium (10 g/l tryptone, 5 g/l yeast extract, 5 g/l NaCl, 15 g/l agar (SIGMA Co.), 50 μg/ml kanamycin (SIGMA Co.), pH 7.5) separately. After shaking at 37° C. overnight, the plasmid contained are isolated with Plasmid Isolating Kit. (Watson Biotechnologies, Inc) according to the standard manual. The positive clones are identified by PCR with above isolated plasmids as the templates.

PCR reaction mixture: 0.1˜1 μl plasmid DNA (about 20-200 ng); 10 μmol upstream primer (T7-up: 5′-TAATACGACTCACTATAGGGGA-3′) (SEQ ID NO:17); 10 μmol down stream primer (T7-down: 5′-GCTAGTTATTGCTCAGCGG-3′) (SEQ ID NO: 18); 2 μl 10× Taq buffer; 2 μl 2 mmol/ml dNTPs; 1U Taq; 12 μl distilled-water. The PCR reaction condition: to pre-denature at 94° C. for 5 minutes; denature at 94° C. for 40 seconds; anneal at 53° C. for 40 seconds; elongate at 72° C. for 40 seconds; 25 cycles. At last 5 μl PCR product are applied to agarose electrophoresis (1%). As shown in FIG. 6, the PCR product is about 500 bp.

### (2). Construction of CD28 VH/pTRI,

The DNA fragment coding anti-CD28VH is amplified from CD28 VH/pTMF (Ju-long et al., 2002)(Cheng et al., 2002) with P1 (P1: 5′-TCACATATGCA GGTACAGC TACAG-3′) (SEQ ID NO: 19) as the up-stream primer and P2 (P2: 5′-TTCGCTAGCGGAAGATACGGTA CCA-3′) (SEQ ID NO: 20) as the down-stream primer. The restriction sites NdeI/NheI are introduced at 5′ end and 3′ end respectively during the process of PCR.

PCR reaction mixture: 1 μl primers (each); 2 μl dNTP (2 mmol/ml each); 2 μl 10× pfu buffer; 100 ng CD28 VH/pTRI, plasmid; 0.3 μl Pfu (Promega Co.); add distilled water to the volume of 20 μl. PCR reaction condition: to pre-denature at 94° C. for 3 minutes; to denature at 94° C. for 30 seconds; to anneal at 55° C. for 30 seconds; to elongate at 72° C. for 50 seconds; 25 cycles. The PCR products of about 350 bp are purified by agarose electrophoresis (1%) and DNA Gel purification kit (Watson Biotech. Inc.).

The above PCR product and pTRI plasmid are cut with NdeI/NheI (Promega Co.) at the same time according to the product manual. The cutting product (about 350 bp) of PCR product and that (about 5300 bp) of pTRI are ligated together and transformed into TOP10 E. coli strain. The plasmids isolated from the positive clones are named as CD28 VH/pTRI, which are identified by PCR with the product of about 750 bp (As shown in FIG. 6). All operating procedures needed here come from step (1).

(3) Construction of CD3 scFv/CD28 VH/pTRI The DNA fragment coding anti-CD3 scFv is amplified from CD3 scFv/pTMF (Liu XF, 1996), with P1 (P1: 5′-AAGAGTACTGAGGTGAAGCTGGTGG-3′) (SEQ ID NO: 21) as the up-stream primer and (P2: 5′-GAAGTCGACAGCGCGCTTCAGTTCCAG-3′) (SEQ ID NO: 22) as the down-stream primer. The restriction sites, ScaI and ScaII, are introduced at 5′ end and 3′ end respectively during the process of PCR.

PCR reaction mixture: 1 μl primers (each); 2 μl dNTP (2 mmol/ml each); 2 μl 10× pfu buffer; 100 ng CD28 VH/pTRI, plasmid; 0.3 μl Pfu (Promega Co.); add distilled water to the volume of 20 μl. PCR reaction condition: to pre-denature at 94° C. for 3 minutes; to denature at 94° C. for 30 seconds; to anneal at 55° C. for 30 seconds; to elongate at 72° C. for 50 seconds; 25 cycles. The PCR products of about 750 bp are purified by agarose electrophoresis (1%) and DNA Gel purification kit (Watson Biotech. Inc.).

The above PCR product and CD28 VH/pTRI plasmid are cut with ScaI/ScaII (Promega Co.) at the same time. The cut PCR product (about 750 bp) and that (about 5700 bp) of CD28 VH/pTRI are ligated together and transformed into TOP 10 E. coli strain. The plasmids isolated from the positive clones are named as CD3 scFv/CD28 VH/pTRI, which are identified by PCR with the product of about 1400 bp (As shown in FIG. 6). All operating procedures needed here come from step (1).

### (4) Construction of CEA-scTsAb/pTRI

Construction of anti-CEA scFv by overlapping PCR:

Anti-CEA scFv is designed by Linking VH (the variable region of heavy chain) and VL (the variable region of light chain) of anti-CEA monoclonal antibody (Koga et al., 1990) with a special polypeptide GGGGSGGGGSGGGGS) (SEQ ID NO: 23). The whole amino acid sequence of anti-CEA scFv is back translated into a DNA sequence according to the E. coli preferred codon table (Nakamura et al., 2000), which is spited into 22 complemental oligo-nucleotides. The 22 oligo-nucleotides listed below are synthesized and assembled into the whole DNA fragment coding anti-CEA scFv by overlapping PCR.

(SEQ ID NO:24)

| | |
|---|---|
| 1. | 5′-TTCCTCGAGCAGGTTCAGCT-3′ |

(SEQ ID NO:25)

| | |
|---|---|
| 2. | 5′-TCGCGCCCGGTTTCATCAGTTCCGCACCGCTCTGCTGCAGCT |
| | GAACCTGCTCGAGGAA-3′ |

(SEQ ID NO:26)

| | |
|---|---|
| 3. | 5′-ACTGATGAAACCGGGCGCGAGCGTGAAAATCAGCTGCAAAGC |
| | GACCGGCTATACCTTC-3′ |

(SEQ ID NO:27)

| | |
|---|---|
| 4. | 5′-CACCCATTCGATCCAATAATCGCTGAAGGTATAGCCGGTCGC |
| | TT-3′ |

(SEQ ID NO:28)

| | |
|---|---|
| 5. | 5′-ATTATTGGATCGAATGGGTGAAACAGCGTCCGGGTCACGGCC |
| | TGGAATGGATCGGTGAA-3′ |

(SEQ ID NO:29)

| | |
|---|---|
| 6. | 5′-ACGTTCGTTGTAGTCGGTACGGCCGCTGCCCGGCAGGATTTC |
| | ACCGATCCATTCCAGG-3′ |

(SEQ ID NO:30)

| | |
|---|---|
| 7. | 5′-CGTACCGACTACAACGAACGTTTCAAAGGCAAAGCGACCTTC |
| | ACCGGCGACGTTTCTAGC-3′ |

(SEQ ID NO:31)

| | |
|---|---|
| 8. | 5′-TTCGCTGGTCAGGCTAGACAGTTTCATATACGCGGTGTTGCT |
| | AGAAACGTCGCCGGTGAA-3′ |

(SEQ ID NO:32)

| | |
|---|---|
| 9. | 5′-TGTCTAGCCTGACCAGCGAAGATAGCGCGGTGTATTACTGCG |
| | CGACCGGCACCACCCCG-3′ |

(SEQ ID NO:33)

| | |
|---|---|
| 10. | 5′-GCTCACGGTCACCAGGGTGCCCTGACCCCAGTAACCGAACGG |
| | GGTGGTGCCGGTCGCGCA-3′ |

(SEQ ID NO:34)

| | |
|---|---|
| 11. | 5′-GCACCCTGGTGACCGTGAGCGCGACTAGTACCCCGAGCCATA |
| | ACAGCCATCAGGTGCCG-3′ |

(SEQ ID NO:35)

| | |
|---|---|
| 12. | 5′-GTCTCTAGAGCCGCTGTTCGCGGTCGGGCCGCCCGCGCTCGG |
| | CACCTGATGGCTGTTAT-3′ |

(SEQ ID NO:36)

| | |
|---|---|
| 13. | 5′-CGAACAGCGGCTCTAGAGACATCGTGCTGACCCAGAGCCCGG |
| | CGAGCCTGGCGGTGTC-3′ |

(SEQ ID NO:37)

| | |
|---|---|
| 14. | 5′-CTGGGAAGCACGGCAGGAGATGGTCGCACGCTGACCCAGAGA |
| | CACCGCCAGGCTCGCCGG-3′ |

(SEQ ID NO:38)

| | |
|---|---|
| 15. | 5′-TCTCCTGCCGTGCTTCCCAGTCCGTTTCCACCTCCTCCTACA |
| | CCTACATGCACTGGTAT-3′ |

(SEQ ID NO:39)

| | |
|---|---|
| 16. | 5′-GATCAGCAGTTTCGGCGGCTGACCCGGTTTCTGCTGATACCA |
| | GTGCATGTAGGTGT-3′ |

(SEQ ID NO:40)

| | |
|---|---|
| 17. | 5′-AGCCGCCGAAACTGCTGATCAAATATGCGAGCAACCTGGAAT |
| | CTGGTGTGCCGGCGCGT-3′ |

(SEQ ID NO:41)

| | |
|---|---|
| 18. | 5′-GTTCAGGGTGAAGTCGGTGCCGCTGCCAGAACCGCTGAAACG |
| | CGCCGGCACACCAGATT-3′ |

(SEQ ID NO:42)

| | |
|---|---|
| 19. | 5′-GCACCGACTTCACCCTGAACATCCACCCGGTGGAAGAAGAAG |
| | ATACCGCGTATTACTAT-3′ |

(SEQ ID NO:43)

| | |
|---|---|
| 20. | 5′-GCCACCGAAGGTACGCGGGATTTCCCAAGAGTGCTGGCAATA |
| | GTAATACGCGGTATCTT-3′ |

(SEQ ID NO:44)

| | |
|---|---|
| 21. | 5′-TCCCGCGTACCTTCGGTGGCGGCACCAAACTGGAAATCAAAG |
| | AATTCGCC-3′ |

(SEQ ID NO:45)

| | |
|---|---|
| 22. | 5′-GGCGAATTCTTTGATTTCCAG-3′ |

(SEQ ID NO:46)

| | |
|---|---|
| S1) | 5′-GGCGAATTCTTTGATTTCCAG-3′ |

(SEQ ID NO:47)

| | |
|---|---|
| S17) | 5′-AGCCGCCGAAACTGCTGATC-3′ |

(SEQ ID NO:48)

| | |
|---|---|
| S16) | 5′-GATCAGCAGTTTCGGCGGCT-3′ |

(SEQ ID NO:49)

| | |
|---|---|
| S13) | 5′-CGAACAGCGGCTCTAGAGAC-3′ |

(SEQ ID NO:50)

| | |
|---|---|
| S12) | 5′-GTCTCTAGAGCCGCTGTTCG-3′ |

(SEQ ID NO:51)

| | |
|---|---|
| S7) | 5′-GTACCGACTACAACGAACGT-3′ |

(SEQ ID NO:52)

| | |
|---|---|
| S6) | 5′-ACGTTCGTTGTAGTCGGTAC-3′ |

The operating steps:

Step 1: according to FIG. 7, to mix the fragments (from 1 to 22) in pair and carry out the elongating reaction without ant primers as below. All products are collected without any purification and applied in next step directly.

Reaction mixture: the synthetic fragments, 1 μl (each); 10× PCR buffer, 2 μl; dNTPs (2 mmol/ml each) (Dalian TaKaRa Biotechnology Co. Ltd.), 2 μl; Taq (1U) (Dalian TaKaRa Biotechnology Co. Ltd.) 0.5 μl; distilled water, 14 μl.

Reaction condition: to pre-denature at 94° C. for 1 minute; to denature at 94° C. for 30 seconds; to anneal at 45° C. for 30 seconds; to elongate at 72° C. for 30 seconds; 10 cycles.

The products are listed: A (1 with 2), B(3 with 4), C(5 with 6), D(7 with 8), E(9 with 10), F(1 with 12), G(13 with 14), H(15 with 16), I(17 with 18), J(19 with 20), K(21 with 22)

Step 2: according to FIG. 7, to mix the products (A, B, D, E, G, H, J, K) of step 1 in pair and carry out the elongating reaction without any primers as below.

Reaction mixture: the products of step 1, 10 μl (each 10 pmol).

Reaction condition: to pre-denature at 94° C. for 1 minute; to denature at 94° C. for 30 seconds; to anneal at 45° C. for 30 seconds; to elongate at 72° C. for 30 seconds; 10 cycles.

All products are applied to agarose electrophoresis (1%) and purified by DNA Gel purifying Kit (Watson Biotech. Inc.). The products are listed: a (A with B), b(C), c(D with E), d(F), e(G with H), f(I), g(J with K). The fragments of “a” and “g” are about 120 bp; the fragments of “c” and “e” are about 170 bp; the fragments of “d” and “f” are about 100 bp.

Step 3: according to FIG. 7, to mix the products of step 2 in pair (a with b, c with d, f with g) or use alone, and carry out the amplifying reaction with respective primers (Primer S1 and S6 correspond to the pair of “a” and “b”; Primer S7 and S112 correspond to the pair of “c” and “d”; Primer S13 and S16 correspond to “e”, Primer S17 and 22 correspond to the pair of “f” and “g”).

Reaction mixture: the product of step 2, 1 μl (each); primers, 1 μl (each); 10× PCR buffer, 2 μl; dNTPs (2 mmol/ml each) (Dalian TaKaRa Biotechnology Co. Ltd.), 2 μl; Taq (1U) (Dalian TaKaRa Biotechnology Co. Ltd.) 0.5 μl; distilled water, 12 μl.

Reaction condition: to pre-denature at 94° C. for 1 minute; to denature at 94° C. for 30 seconds; to anneal at 45° C. for 30 seconds; to elongate at 72° C. for 30 seconds; 25 cycles.

All products are applied to agarose electrophoresis (1%) and purified by DNA Gel purifying Kit (Watson Biotech. Inc.). The products are listed: I (a with b), II (c with d), III (e), IV (f with g). The product I is about 200 bp; the product II is about 250 bp; the product III is about 140 bp; the product IV is about 230 bp.

Step 4: according to FIG. 7, to mix the products of step 3 in pair (I with II, III with IV) and carry out the amplifying reaction with respective primers as below. Primer S1 and S12 correspond to the pair of I and II; Primer S13 and 22 correspond to the pair of III and IV.

Reaction mixture: the product of step 3, 1 μl (each); primers, 1 μl (each); 10× PCR buffer, 2 μl; dNTPs (2 mmol/ml each) (Dalian TaKaRa Biotechnology Co. Ltd.), 2 μl; Taq (1U) (Dalian TaKaRa Biotechnology Co. Ltd.) 0.5 μl; distilled water, 12 μl.

Reaction condition: to pre-denature at 94° C. for 1 minute; to denature at 94° C. for 30 seconds; to anneal at 45° C. for 30 seconds; to elongate at 72° C. for 30 seconds; 25 cycles.

All products are applied to agarose electrophoresis (1%) and purified by DNA Gel purifying Kit (Watson Biotech. Inc.). The fragment of UP is about 430 bp; the fragment of DOWN is about 340 bp. The products are listed: UP (I with II), DOWN (III with IV).

Step 5: according to FIG. 7, to mix the products (UP and DOWN) of step 4 in pair and carry out the amplifying reaction with primer S1 and 22.

Reaction mixture: the product of step 4, 1 μl (each); primers, 1 μl (each); 10× PCR buffer, 2 μl; dNTPs (2 mmol/ml each) (Dalian TaKaRa Biotechnology Co. Ltd.), 211; Taq (1U) (Dalian TaKaRa Biotechnology Co. Ltd.) 0.5 μl; distilled water, 12 μl.

Reaction condition: to pre-denature at 94° C. for 1 minute; to denature at 94° C. for 30 seconds; to anneal at 45° C. for 30 seconds; to elongate at 72° C. for 60 seconds; 25 cycles.

The product is applied to agarose electrophoresis (1%) and purified by DNA Gel purifying Kit (Watson Biotech. Inc.). The product WHOLE is about 750 bp.

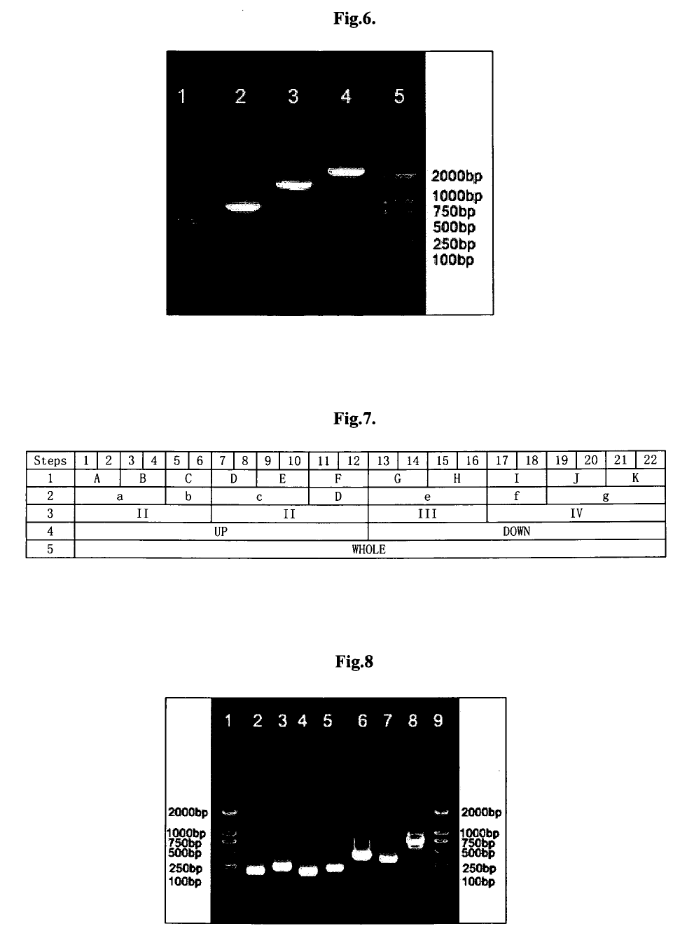

The above schematic process of above operations is shown in FIG. 7 and the results of identifying PCR is shown in FIG. 8.

The above PCR product and pTMF plasmid are cut with XhoI/EcoRI (Promega Co.) at the same time. The cutting product (about 750 bp) of PCR product and that (about 5200 bp) of pTMF are ligated together and transformed into TOP10 E. coli strain. The plasmids isolated from the positive clones are named as CEA scFv/pTMF, which are identified by PCR with the product of about 750 bp. All operating procedures needed here come from step (1).

The CEA scFv/pTMF plasmid and the CD3 scFv/CD28 VH/pTRI plasmid are cut with XhoI/EcoRI (Promega Co.) at the same time. The small cut product (about 750 bp) of the former and large one (about 6000 bp) of the latter are ligated together and transformed into TOP10 E. coli strain. The plasmids isolated from the positive clones are named as CEA scTsAb/pTRI, which are identified by PCR with the product of about 2100 bp (As shown in FIG. 6). All operating procedures needed here come from step (1).

### Example 3

### Soluble Cytoplasmic Expression of CEA-scTsAb Induced at Lower Temperature

(1) Transformation of CEA scTsAb/pTRI into BL21 (DE3)(Novagen) E. coli Strain.

The competent BL21 (DE3) cells are prepared referring to the method in example 2. The plasmid CEA scTsAb/pTRI) is isolated with plasmid isolating kit (Watson Biotech. Inc.) according to the manual. The subsequent procedures of transformation and identification of positive clones are performed according to example 2 too.

### (2) Induced Expression at Lower Temperature

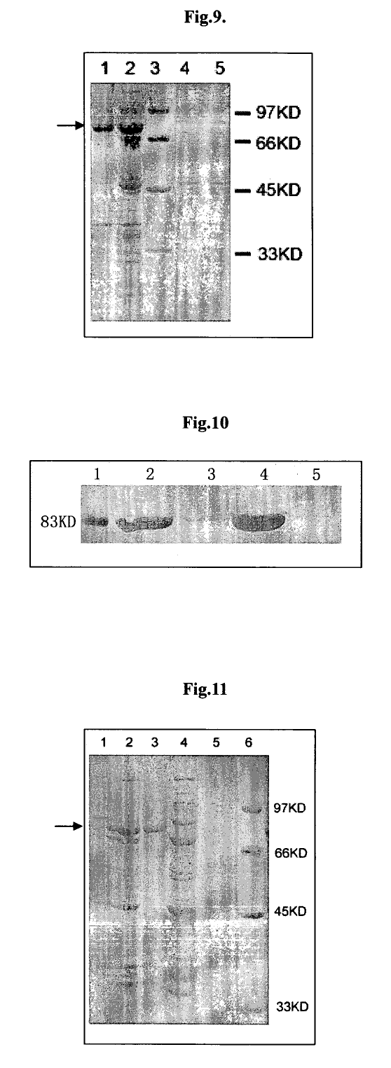

The single clone of BL21 (DE3) containing CEA-scTsAb/pTRI is pick up from LB-K plate and inoculated in 5 ml LB-K medium. After being cultured at 37° C. with shaking overnight, the culture is transferred into 250 ml LB-K medium at a ratio of 1/100 to shake at 37° C. to reach A600 0.6. IPTG (Takara Biotech. (Dalian)) is added to the final concentration of about 0.4 mmol/l to induce soluble expression at 30° C. for 4 hours. The bacterial cells are harvested by centrifuging at 12,000 rpm for 10 minutes and then re-suspended in phosphate buffered saline (PBS: 8 g NaCl, 0.2 gKCl, 1.44 g Na₂HPO₄, 0.24 g KH₂PO₄, pH7.4, 1 liter) (1/5 volume of culture medium). Thus, cytoplasmic soluble CEA-scTsAb is released into the supernatant produced by centrifuging ultrasonic-lyzed cells. Furthermore, soluble expression and inclusion body expression of CEA-scTsAb are detected by reducing SDS-PAGE and Western-blotting according to “molecular cloning: a laboratory manual” (Translated by Jin Dong-yan and Li Meng-feng, 1996, Science Press in China)(Sambrook and Russell, 2001). The expression of CEA-scTsAb in both supernatant and pellet from sonication are detected by SDS-PAGE and Western blot and photographed with Alpha-Image 2200 Documentation and analysis system (American Alpha Innotech Company). As shown in FIG. 9 and FIG. 10, adopting above soluble cytoplasmic expressing method, the soluble CEA-scTsAb occupies about 70% of its total expression. As ultrasonic supernatant can be applied in further steps of purification and in vitro activity assay directly, in no need of denaturation or renaturation, the cost and time of production would be saved remarkably.

### Example 4

### Purification of CEA-scTsAb by DEAE Anion Exchange Chromatography

250 ml culture medium containing bacterial cells expressing CEA-scTsAb are centrifuged at 12,000 rpm at 4° C. for 10 minutes. The pellet is suspended in 50 ml equilibrium buffer (20 mmol/l NaCl, 20 mmol/1 Tris-HCl, pH 8.0) of DEAE anion exchange chromatography for further sonication. After a second centrifugation at 12,000 rpm at 4° C. for 10 minutes, the supernatant containing solubly expressed CEA-scTsAb is applied in purifying step directly.

20 ml of DEAE anion exchange resin (Amersham Bioscience) is suspended in 100 ml equilibrium buffer and packed into a 16×20 cm column (Shanghai Hua-mei). The column is equilibrated with 5 volume of equilibrium buffer at a velocity of 1 ml/minute. The above supernatant is then loaded at a velocity of 0.25 ml/minute. Purified CEA-scTsAb exists in the flow-through fraction. The column is washed or eluted with 2 volume of eluting buffer (500 mmol/I NaCl, 20 mmol/l Tris-HCl, pH 8.0) at a velocity of 0.25 ml/minute and cleaned with 2 volume of 500 mmol/I NaOH at a velocity of 0.5 ml/minute. During the regenerating step, the column is first washed with 2 volume of 1000 mmol/ml NaCl at a velocity of 0.5 ml/minute, and then equilibrated with 2 volume of equilibrium buffer at a velocity of 1 ml/minute for next cycle of purification. At least 4 volume of 20% ethanol should be used to wash resins in avoiding of contamination before storage of the column.

By reducing SDS-PAGE of above flow through fraction, the result of purification is shown in FIG. 11. As a result, most of bacterial protein in the supernatant can be removed by a single step of DEAE anion exchange chromatography, and CEA-scTsAb occupied 70% of the flow-through fraction, quantified with digital image analyzer (Alpha-Image 2200 Documentation and analysis system (American Alpha Innotech Company)

The purified sample is then dialyzed against PBS at 4° C. overnight, changing dialyzing buffer every 6 hours. The protein concentration is quantified with Bradford method from “Short protocols in molecular biology: a compendium of methods from Current protocols in molecular biology” (Translated by Yan Zhi-ying and Wang Hai-lin, Revised by Jin Dong-yan, 1999, Science Press in China)(Ausubel, 1999). Then, after supplementing sodium azide (0.05% (W/V), Sigma) as the preservative and trehalose (0.15 mol/l, from Microbiology Institute, Chinese Academy of China) as the stabilizer, the product solution is divided into 1 ml aliquot and stored at −80° C.

### Example 5

### Detection of the Binding Specificity to Three Antigens (CEA, CD3, CD28) by ELISA

Preparation of Jurkat cell membrane antigen: 5×10⁶ Jurkat cells (American type culture collection, ATCC, TIB-152) are harvested by centrifuging at 1000 g for 10 minutes. The cell pellet is suspended in 0.5 ml PBS and lysed by ultra-sonication. The supernatant of ultra-sonication produced by centrifuging at 12,000 rpm for 10 minutes is supplemented with sodium azide (0.05% (W/V), Sigma) and trehalose (0.15 mol/l, from Microbiology Institute, Chinese Academy of China), divided into 100 μl aliquots and stored at −80° C.

### ELISA:

(1) Coating: purified CEA (Fitzerald, German), rhCD28-FC chimera(R&D) and above purified Jurkat membrane antigen are diluted in coating buffer (1.36 g Na₂CO₃, 7.35 g NaHCO₃, 1 liter, pH 9.2) in the concentration of 1 μg/ml (CEA and rhCD28-FC chimera) or 10 μg/ml (Jurkat membrane antigen) and coated with 100 μl/well in 96 well ELISA plate (Nunc). The plate is placed at 37° C. for 2 hours or at 4° C. overnight.
(2) Blocking: the coating plate is washed with PBS for 1-2 times and the blocking buffer (PBS-1% BSA (Bovine Serum Albumin, w/v) is added with 200 μl/well. The plate is placed at 37° C. for 2 hours.
(3) Addition of Samples: the blocked plate is washed with PBS for 3 times and the diluted sample in PBS is added in triplicate with 100 μl/well. The samples of CEA-scTsAb are semi-diluted from the primary concentration of 10 μg/ml for 6 times. The plate is placed at 37° C. for 2 hours.
(4) Addition of the primary antibody: the plate is washed with PBS-T (PBS-0.05% Tween-20 (w/v)) for 3 times and 1/1000 diluted mouse anti-cmyc tag monoclonal antibody (Santa Crutz) is added with 100 μl/well. The plate is placed at 37° C. for 2 hours.
(5) Addition of the secondary antibody: the plate is washed with PBS-T for 3 times and 1/1000 diluted HRP (horse-radish peroxidase) conjugated goat anti-mouse IgG (Santa Crutz) is added with 100 μl/well. The plate is placed at 37° C. for 2 hours.
(6) Visualization: the secondary antibody in the plate is washed with PBS-T for 5 times and the visualizing solution containing 10 ml substrate buffer (36.6 g Citric Acid, monohydrate, 113.5 g Potassium dibasic phosphate, 1 liter, pH6.0) and 4 mg OPD (orthofenylenediamin.diHCl, Sigma) is added with 100 μl/well. The plate is placed at room temperature in the dark for 20 minutes.
(7) Stop reaction: 1 mol/l HCl is added with 100 μl/well to stop the reaction.
(8) Measurement: the absorbent result is read at 490 nM.

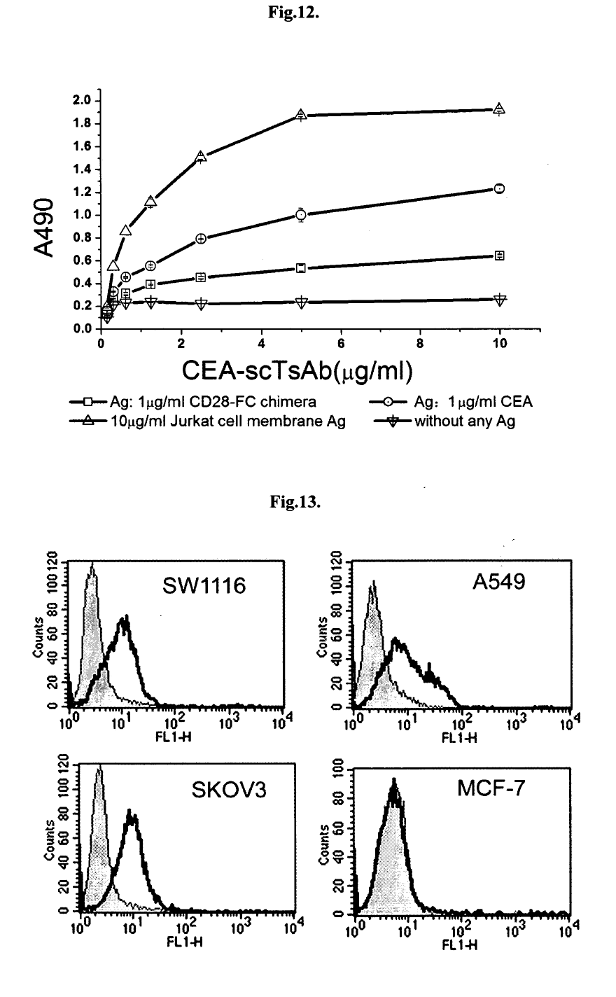

As shown in FIG. 12, CEA-scTsAb binds to two pure antigens (CEA, rhCD28-FC chimera) very specially. As CD3 is expressed on Jurkat cell abundantly, CEA-scTsAb also binds to Jurkat membrane antigen specially.

### Example 6

### Detection of the Binding Specificity to Tumor Cells by FACS

An indirect FACS method is used to detect the binding to various tumor cells. The sources of these tumor cells are listed below.

| Designation | Source | ATCC Number |
|---|---|---|
| A549 | Lung, Carcinoma | CCL-185 |
| MCF-7 | Human, Mammary Gland, | HTB-22 |
| | Breast Adenocarcinoma | |
| SK-OV-3 | Ovary, Adenocarcinoma | HTB-77 |
| SW1116 | Colorectal Adenocarcinoma | CCL-233 |

### Operating:

(1) Culture and collection of tumor cells: three types of tumor cells (A549, MCF-7, SK-OV-3) are cultured in 10% fetal calf serum (FCS, Hei-Long-Jiang-Jiang-Hai Bioengineering technology Co.)-containing RPMI 1640 medium (Gibco) plus antibiotics (100 U penicillin) in humidified 5% CO₂ incubator at 37° C. SW1116 is cultured in 10% fetal calf serum (FCS, Hei-Long-Jiang-Jiang-Hai Bioengineering technology Co.)-containing L15 medium (Gibco) plus antibiotics (100 U penicillin) in humidified 5% CO₂ incubator at 37° C. 5×10⁵ tumor cells in the exponential phase are collected by centrifuging at 1000 g for 10 minutes and suspended in 100 μl PBS.
(2) After another centrifugation of tumor cells at 1000 g for 10 minutes and suspending in 100 μl PBS, CEA-scTsAb is added to the final concentration of 10 μg/ml. The isotype control is set for each tumor cell. The cell suspensions are incubated at 4° C. for 30 minutes.
(3) Incubation of, with tumor cells: after centrifuging at 1000 g for 10 minutes, unbound CEA-scTsAbs are removed by discarding the supernatant, and the cell pellet is suspended in 100 μl PBS containing 1/1000 diluted primary antibody (Santa Crutz). The cell suspensions are incubated at 4° C. for 30 minutes.
(4) The unbound primary antibodies are removed by discarding the supernatant after centrifuging at 1000 g for 10 minutes. The cell pellet is suspended in 100 μl PBS containing 1/1000 diluted secondary antibody. The cell suspensions are incubated at 4° C. for 30 minutes.
(5) FACS analysis: the unbound secondary antibodies are removed by discarding the supernatant after centrifuging at 1000 g for 10 minutes. The cell pellet is suspended in 400 μl PBS and analyzed with FACS Calibur (BD). The excitating light wave is 488 nM. 10,000 cells are collected every time.

As shown in FIG. 13, CEA-scTsAb binds to SW1116 and SK-OV-3 best of all; CEA-scTsAb binds to A549 modestly; CEA-scTsAb does not bind to MCF-7.

### Example 7

### FACS Analysis of the Binding Specificity of CEA-scTsAb to PBMC and Jurkat Cells

A direct FACS method is used here to test the binding specificity of CEA-scTsAb to PBMC (From Beijing Blood Bank) and Jurkat cells.

### Operating:

(1) Conjugation of FITC (Sigma) to CEA-scTsAb: FITC is conjugated to CEA-scTsAb with the “Clark method” according to “Xian Dai Mian Yi Xue Shi Yan Ji Shu” (Edited by Shen Guan-Xin and Zhou Ru-lin, HuBei Science Technology Press, 2002)(Guan-xin and Ru-lin, 2002)
(2) PBMC are prepared by Ficol gradient centrifugation and cultured in 10% fetal calf serum (FCS)-containing RPMI1640 medium (Gibco) plus antibiotics (100 U penicillin) in humidified in humidified 5% CO 2 incubator at 37° C. After being incubated for 4 hours, the suspended cells, mainly lymphocytes, are transferred to a new flask. Thus the adherent cells are removed. The Ficol centrifugation is also performed according to “Xian Dai Mian Yi Xue Shi Yan Ji Shu” (Edited by Shen Guan-Xin and Zhou Ru-lin, HuBei Science Technology Press, 2002)(Guan-xin and Ru-lin, 2002)
(3) Both PBMC and Jurkat cells are cultured in 10% fetal calf serum (FCS)-containing RPMI1640 medium (Gibco) plus antibiotics (100 U penicillin) in humidified 5% CO₂ incubator at 37° C.
(4) 5×10⁵ PBMC or Jurkat cells in the exponential phase are collected by centrifuging at 1000 g for 10 minutes and suspended in 100 μl PBS, which contains 10 μg/ml FITC conjugate of CEA-scTsAb. The isotype control is set for each tumor cell. The cell suspensions are incubated at 4° C. for 30 minutes.
(5) After another centrifugation at 1000 g for 10 minutes. The cell pellet is re-suspended in 400 μl PBS and analyzed with FACS Calibur (BD). The excitating light wave is 488 nM. 10,000 cells are collected every time.

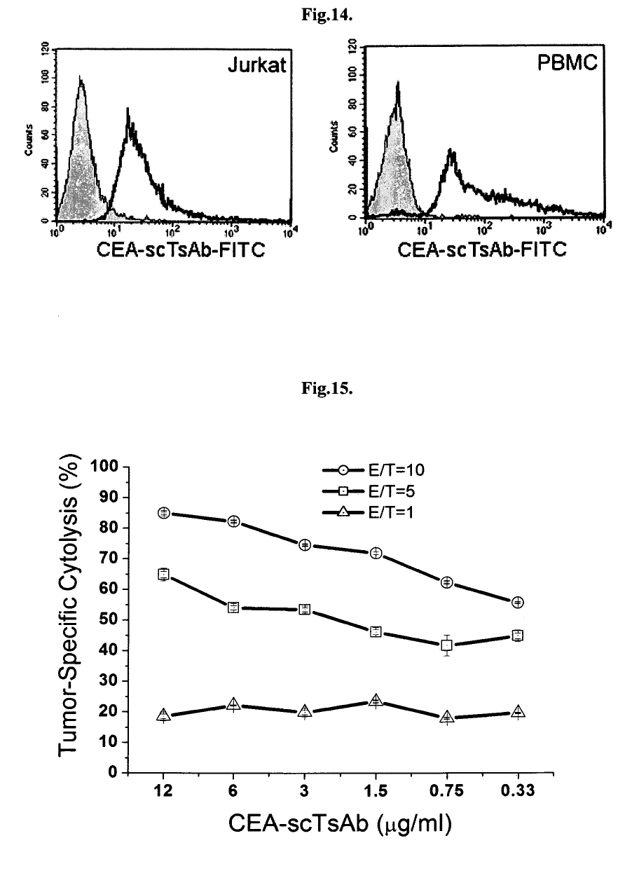

As shown in FIG. 14, CEA-scTsAb binds to PBMC and Jurkat cells specially.

In summary of example 6 and example 7, CEA-scTsAb could bind to PBMC, Jurkat, and several CEA expressing tumor cells specially.

### Example 8

### Detection of Tumor Specific Cytolysis of Colorectal Carcinoma Cell, SW1116, induced by CEA-scTsAb in the Presence of Lymphocytes with MTT Assay

In the system of in vitro assay of tumor specific cytolysis, CEA expressing tumor cell line, SW1116, is used as the target cell (T), and lymphocytes from PBMC is used as the effector cell (E). After mixing them together at a certain ratio of E/T, CEA-scTsAb is added, tumor specific cytolysis is induced by incubating at 37° C. for 48 hours. The survival level of tumor cells is then tested with MTT assay to evaluate tumor specific cytolysis.

(1) PBMC are prepared according to example 7.
(2) SW1116 cells are cultured and collected according to example 6.
(3) Diluted in 10% FCS containing L15 medium (Gibco Co), SW1116 cells (1×10⁵/ml) are first plated in 96-wells plate (Nunc) with 100 μl/well. Then effector cells (PBMC) are added at different E/T ratio (1, 5, 10) with 100 μl/well. Concentrated CEA-scTsAbs (5 μg/ml) are supplemented with 50 μl/well to reach a final concentration of 1 μg/ml, which is also diluted in 10% FCS containing LI 5 medium (Gibco Co). The mixture is incubated at 37° C. 5% CO₂ incubator for about 48 hours. Quadruplicate wells are set for each concentration. The setting of negative controls: no CEA-scTsAb wells for each E/T ratio; the wells containing effector cells only; the wells containing target cells only; the wells containing no cells.
(4) MTT assay: the medium supernatants are removed by aspirating, and the adherent cells are washed with PBS one time. Add 200 μl MTT solution (MTT: (3-(4, 5-dimethylthiazolyl-2)-2,5-diphenyltetrazolium bromide, 500 μg/ml, Sigma) for each well and incubate at 37° C. for 4 hours. Wash the plate one time with PBS and add 200 μl DMSO (Sigma) for each well. Continue to incubate at 37° C. for 30 minutes. Absorbance of each well is measured at a wavelength of 570 nm with background subtraction at 620 nm.
(5) The percent of tumor specific cytolysis is calculated according to the formula:

The percent of tumor specific cytolysis(%)=[A600(ET)−A600(ETA)]/[A600(ET)−A600(M)]×100%

A600(ET): the absorbance of the negative wells without CEA-scTsAb.

A600(ETA): the absorbance of the sample wells.

A600(M): the absorbance of the negative wells containing no cells.

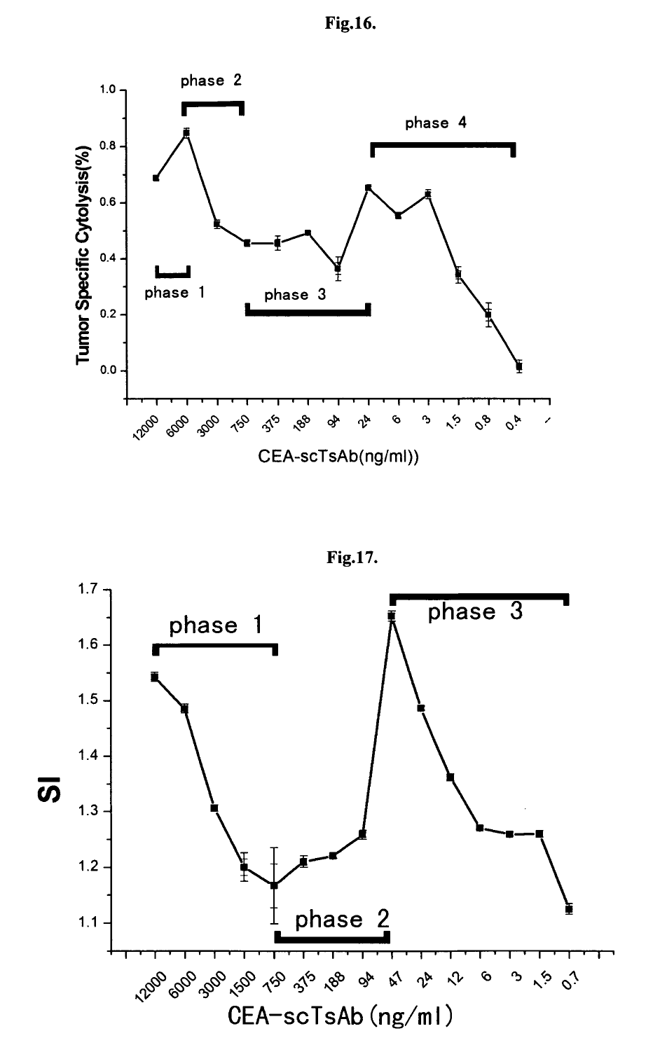

The effect of E/T ratio on tumor specific cytolysis induced by CEA-scTsAb is shown in FIG. 15. It can be concluded that there is no direct correlation between E/T ratio and the efficiency of tumor specific cutolysis. It is lowest at E/T ratio 1, moderate at E/T ratio 10, and highest at E/T ratio 5. So E/T ratio 5 is the optimal ratio, at which tumor specific cytolysis reaches 85%. It also suggests that there are other affecting factors for tumor specific cytolysis except E/T ratio. Fixing the E/T ratio at 5, the effect of increasing the concentration of CEA-scTsAb from 0.4 ng/ml to 12 μg/ml on tumor specific cytolysis is shown in FIG. 16. The curve displays a four-stepwise phases for tumor specific cytolysis. In the first phase from 61 g/ml to 12 μg/ml, the efficiency of tumor specific cytolysis displays negative correlation with the concentration of CEA-scTsAb and reach the peak at 6 μg/ml; In the second phase from 750 ng/ml to 6 μg/ml. it displayed a direct correlation and reach the bottom at 750 ng/ml; In the third phase from 24 ng/ml to 750 ng/ml, it turn back into negative correlation; In the fourth phase from 24 ng/ml to zero, the direct correlation appeared again. Anyhow, two peak of tumor specific cytolysis exist: 85% at 12 μg/ml; 70% at 24 ng/ml. It can be concluded from above data that extremely efficient tumor specific cytolysis could be induced even at lower E/T ratio or lower concentration of CEA-scTsAb.

### Example 9

### Morphological Observation of Tumor Cells During the Process of Tumor Specific Cytolysis Induced by CEA-scTsAb

After mixing PBMC (effector cells) with SW1116 cells (target cells) in L15 medium (10% FBS) at an E/T ratio of 5, and adding purified CEA-scTsAb at a concentration of 750 ng/ml, the mixture is incubated at 37° C. for 20-40 h in 5% CO₂ incubator. Then morphological changes of tumor cells and PBMC are observed under a 40× object lens with an OLYMPUS IMT-2 inverted microscope, and recorded by photomicrography. As shown in FIG. 18, there are four steps of morphological changes. At first, target cells fall off from the plate continuously (FIG. 18 (B)); Then effector cells gathered on their surface (FIG. 18 (C)); The target cell membrane become protuberant with the accumulation of effector cells (FIG. 18 (D)); At last, the boundary of target cells become dimness and target cells break up to death (FIG. 18 (E,F,G)).

### Example 10

### Detection of the Proliferation of Effector Cells Incubated with Target Cells and CEA-scTsAb with MTT Assay

The proliferation of effector cells (mainly T lymphocytes) detected with MTT assay is used to evaluate the activation of T lymphocytes induced by co-incubated tumor cells and CEA-scTsAb.

### Operations:

(1) PBMC are prepared according to example 6.
(2) SW1116 tumor cells are cultured and collected according to example 6 too.
(3) Adjust the concentration of SW1116 cells to about 10⁶/ml in mitomycin C containing (25 μg/ml Sigma) L15 medium and incubate the cell mixture at 37° C. 5% CO2-incubator for 20 minutes. After washing the tumor cells with PBS three times, residual mitomycin C is removed from culture medium.
(4) Adjust the concentration of SW1116 cells to 10⁵/ml and that of PBMC to 5×10⁵/ml in 10% FCS containing L15 medium. Concentrated CEA-scTsAbs (5 g/ml) are supplemented with 50 μl/well to reach a final concentration of 1 μg/ml, which is also diluted in 10% FCS containing L15 medium (Gibco Co). Plate 100 μl of them in 96-well plate and incubate the cell mixture at 37° C. 5% CO₂ incubator for 4 days. Quadruplicate wells are set for each concentration of CEA-scTsAb. Negative controls: no CEA-scTsAb wells for each E/T ratio; the wells containing effector cells only; the wells containing target cells only; the wells containing no cells.
(5) MTT assay: the medium supernatants are removed by aspirating, and the adherent cells are washed with PBS one time. Add 200 μl MTT solution (MTT: (3-(4, 5-dimethylthiazolyl-2)-2,5-diphenyltetrazolium bromide, 500 μg/ml, Sigma) for each well and incubate at 37° C. for 4 hours. Wash the plate one time with PBS and add 200 μl DMSO (Sigma) for each well. Continue to incubate at 37° C. for 30 minutes. Absorbance of each well is measured at a wavelength of 570 nm with background subtraction at 620 nm.
(6) The stimuli index (SI) is calculated according to the formula below:

SI=[A600(ETA)−A600(ET)]

A600(ET): the absorbance of the negative wells without CEA-scTsAb.

A600(ETA): the absorbance of the sample wells.

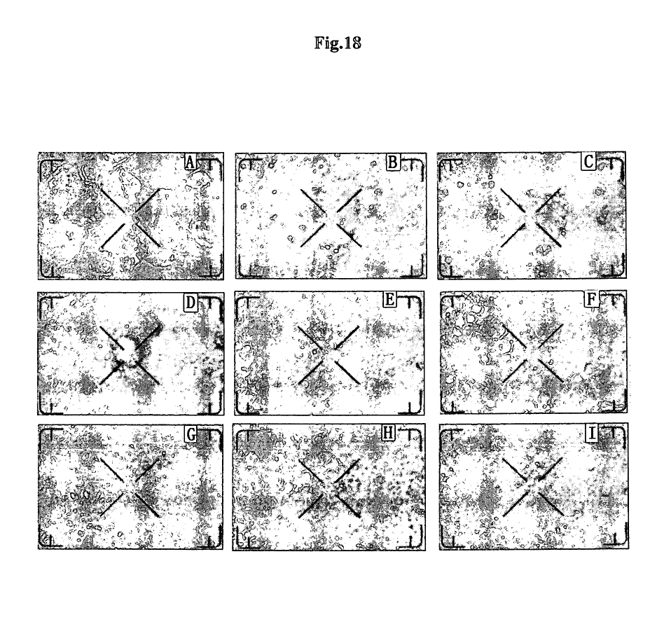

As shown in FIG. 17, There are three stepwise phases for stimuli index (SI). In the first phase from 1.5 μg/ml to 12 μg/ml, SI displays direct correlation with the concentration of CEA-scTsAb and reach the bottom at 6 μg/ml; In the second phase from 50 ng/ml to 6 μg/ml. it displayed a negative correlation and reach the peak at 50 ng/ml; In the third phase from 50 ng/ml to zero, it turn back into direct correlation. It can be concluded that CEA-scTsAb possess of the simulating ability to T lymphocytes even at extremely lower concentration. It is also found that tumor specific cytolysis induced by CEA-scTsAb correspond to the activating state of co-incubated T lymphocytes.

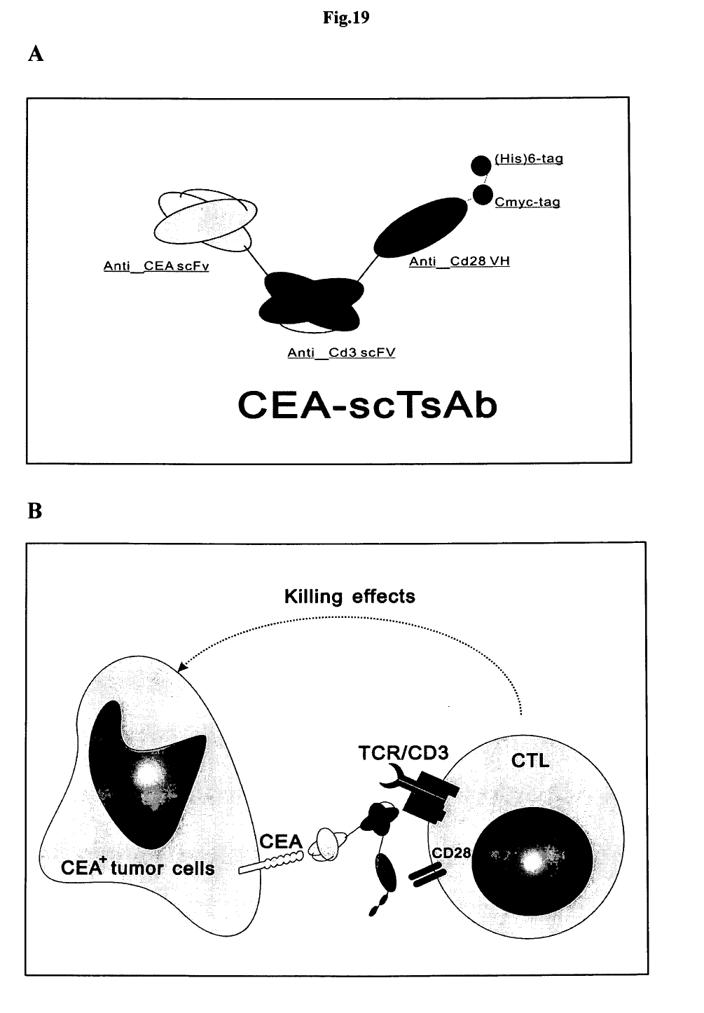

To sum up the results from example 4-9, the function CEA-scTsAb focus on two aspects: (1) retargeting effector cells around tumor cells; (2) stimulating effector cells to kill target cells specially. As summarized in FIG. 19, retargeted cytotoxic T lymphocytes (CTL) are activated to kill target tumor cells directly. T helper cells secret cytokines, such as IL-2, IFN-γ and TNF-α, to assist CTL or natural killing cells (NK cell) in killing target tumor cells indirectly.

### Example 11

### The Mechanism of Tumor Specific Cytolysis Induced by CEA-scTsAb

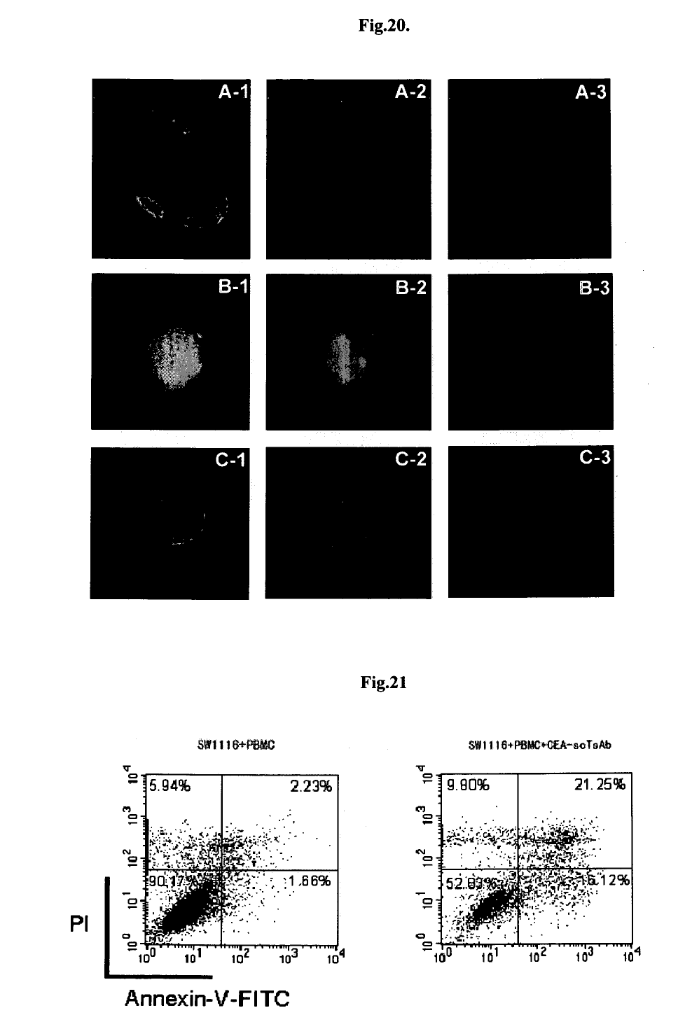

There are three pathway for activated CTLs to kill tumor cells in vivo: activated CTLs secret performs to make holes on the membrane surface of tumor cells, which are broken up and induced to necrosis; grazymes secreted by activated CTLs can enter tumor cells through above holes and induce apoptosis; acitivated CTLs would be induced to express Fas ligands on its surface, which interact with Fas molecules on tumor cells and induce them to apoptosis. PI/annexin-V-FITC dual-color FACS (fluorescence cytometry) and subsequent fluorescence microphotography are used here to distinguish necrosis from apoptosis of tumor cells in in vitro assay of tumor specific cytolysis.
(1) PBMC are prepared according to example 7.
(2) SW1116 cells are cultured and collected according to example 6.
(3) 104 SW1116 cells in 10% FCS containing L15 medium are first plated in 48-wells plate (Nunc) for each well. Then effector cells (PBMC) are added at an E/T ratio of 5. Concentrated CEA-scTsAbs (5 μg/ml) are supplemented with 50 μl/well to reach a final concentration of 1 μg/ml, which is also diluted in 10% FCS containing L15 medium (Gibco Co) The mixture is incubated at 37° C. 5% CO₂ incubator for about 20 hours. Quadruplicate wells are set for each concentration. Negative controls: no CEA-scTsAb wells for each E/T ratio; the wells containing effector cells only; the wells containing target cells only.
(4) After centrifugation at 1000 g for 10 minutes, the supernatants are discarded and trypsin(0.3%, Sigma) containing RPMI1640 medium is added with 50 μl/well. After 1 minute, fresh 10% FCS containing medium is added with 1 ml/well to allow gently suspending digested cells the cell suspension for each well is transferred into 1.5 ml tubes and centrifuge at 1000 g for 10 minutes.
(5) After being Washed with PBS one time, the cell pellet for each well prepared by centrifuging at 1000 g for 10 minutes are then suspended in 100 μl binding buffer (BD) and incubated with 5 μl FITC conjugate of Annexin-V (BD) and 5 μl PI solution (Sigma, 50 μg/ml). The mixture is incubated at room temperature in dark for 15 minutes.
(6) After being supplemented with 300 μl binding duffer, a small part of cells are photographed under fluorescence microscope (Leica DMRA2) and analyazed with QFISH Software (Leica). The reresults are shown in FIG. 20.
(7) Other diluted cells are analyzed by dual-color FACS (FACS Calibur, BD) directly. The excitating wavelength is 488 nM. 20,000 cells are collected for each vial. The reresults are shown in FIG. 21.

As shown in FIG. 20, early apoptosis, late apoptosis and necrosis are distinguished with two dyes: early apoptosis cells are dyed with green fluorescence (FITC conjugate of annexin V) only; late apoptosis cells are dyed with both of them; necrosis cells are mainly dyed with red fluorescence (PI) with weak green fluorescence.

In FIG. 21, four quadrants represent four states of tumor cells: the up left quadrant (UL) is necrosis cells; the up right quadrant (UR) is late apoptosis cells; the low left quadrant is live cells; the low right quadrant is early apoptosis cells. The representative results are shown in FIG. 21. Negative controls without CEA-scTsAb: LL is 90.17%; LR is 1.66; UR is 2.23%; UL is 5.94%. Sample wells: LL is 52.83%; LR is 16.12%; UR is 21.25%; UL is 9.80%. It can be concluded that tumor specific cytolysis induced by CEA-scTsAb be attributed to both necrosis and apoptosis of tumor cells. Compared with negative control, both early apoptosis and late apoptosis are increased 9 times, while necrosis is increased 2 times.

What is claimed is:

1.-20. (canceled)

21. A single chain tri-specific antibody comprising in tandem an anti-tumor associated antigen antibody fragment, a first interlinker, an anti-CD3 antibody fragment, a second interlinker and an anti-CD28 antibody fragment.

22. The single chain tri-specific antibody of claim 21 further comprising (i) a c-myc tag (SEQ ID NO: 56), (ii) a (His)6 tag (SEQ ID NO: 57), or (iii) a c-myc tag (SEQ ID NO: 56 and a (His)6 tag (SEQ ID NO: 57), in the carboxy terminal region of said single chain tri-specific antibody.

23. The single chain tri-specific antibody of claim 21, wherein said first interlinker is a Fc linker comprising an amino acid sequence set forth in SEQ ID NO: 53.

24. The single chain tri-specific antibody of claim 21, wherein said second interlinker is a HSA linker comprising an amino acid sequence set forth in SEQ ID NO: 54.

25. The single chain tri-specific antibody of claim 21, wherein said anti-tumor associated antigen antibody fragment is an anti-carcinogenic embronic antigen antibody fragment.

26. The single chain tri-specific antibody of claim 21, wherein said anti-tumor associated antigen antibody fragment is a single chain variable fragment (scFv), a Fab fragment, or a variable region of the heavy chain, of an anti-tumor associated antigen antibody.

27. The single chain tri-specific antibody of claim 21, wherein said anti-tumor associated antigen antibody fragment comprises the amino acid sequence set forth in SEQ ID NO: 1.

28. The single chain tri-specific antibody of claim 21, wherein said anti-tumor associated antigen antibody fragment is a single chain variable fragment (scFv), a Fab fragment, or a variable region of the heavy chain, of an anti-CD3 antibody.

29. The single chain tri-specific antibody of claim 21, wherein said anti-CD3 antibody fragment comprises the amino acid sequence set forth in SEQ ID NO: 2.

30. The single chain tri-specific antibody of claim 21, wherein said anti-tumor associated antigen antibody fragment is a single chain variable fragment (scFv), a Fab fragment, or a variable region of the heavy chain, of an anti-CD28 antibody.

31. The single chain tri-specific antibody of claim 21, wherein said anti-CD28 antibody fragment comprises the amino acid sequence set forth in SEQ ID NO: 3.

32. The single chain tri-specific antibody of claim 21 comprising the amino acid sequence of SEQ ID NO: 4.

33. The single chain tri-specific antibody of claim 21, wherein said antibody is purified.

34. A DNA sequence encoding the single chain tri-specific antibody of claim 21.

35. The DNA sequence of claim 33, comprising the nucleic acid sequence of SEQ ID NO: 3.

36. An expression vector comprising the DNA sequence of claim 33.

37. The expression vector of claim 35 comprising the DNA sequence of claim 33 and pTRI.

38. A host cell comprising the expression vector of claim 35.

39. A method for producing a single chain tri-specific antibody comprising culturing the host cell of claim 37 such that the DNA sequence encoding the single chain tri-specific antibody is expressed, and purifying said single chain tri-specific antibody.

40. A method for treating or preventing a cancer that expresses carcinogenic embryonic antigen, said method comprising administering a pharmaceutical composition comprising a purified single chain tri-specific antibody of claim 25.


SEQUENCE LISTING (58 sequences)

SEQ ID NO: 1 - protein, 251 aa, Murine
  murine anti-CEA single chain fragment of variable region contained in CEA-scTsAb
QVQLQQSGAELMKPGASVKISCKATGYTFSDYWIEWVKQRPGHGLEWIGEILPGSGRTDYNERFKGKATFTGDVSSNTAYMKLSSLTSEDSAVYYCATGTTPFGYWGQGTLVTVSATSTPSHNSHQVPSAGGPTANSGSRDIVLTQSPASLAVSLGQRATISCRASQSVSTSSYTYMHWYQQKPGQPPKLLIKYASNLESGVPARFSGSGSGTDFTLNIHPVEEEDTAYYYCQHSWEIPRTFGGGTKLEIK

SEQ ID NO: 2 - protein, 250 aa, Murine
  murine anti-CEA single chain fragment of variable region contained in CEA-scTsAb
EVKLVESGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTSVTVSSTSGGGGSGGGGSGGGGSSRDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLELKRA

SEQ ID NO: 3 - DNA, 2103 bp, Artificial Sequence
  CEA-scTsAb sequence
atgggtctcgagcaggtgcagctgcagcagagcggtgcggaactgatgaaaccgggcgcgagcgtgaaaatcagctgcaaagcgaccggctataccttcagcgattattggatcgaatgggtgaaacagcgtccgggtcacggcctggaatggatcggtgaaatcctgccgggcagcggccgtaccgactacaacgaacgtttcaaaggcaaagcgaccttcaccggcgacgtttctagcaacaccgcgtatatgaaactgtctagcctgaccagcgaagatagcgcggtgtattactgcgcgaccggcaccaccccgttcggttactggggtcagggcaccctggttaccgtttccgcgactagtaccccgagccataacagccatcaggtgccgagcgcgggcggcccgaccgcgaacagcggctctagagacatcgtgctgacccagagcccggcgagcctggcggtgtctctgggtcagcgtgcgaccatctcctgccgtgcttcccagtccgtttccacctcctcctacacctacatgcactggtatcagcagaaaccgggtcagccgccgaaactgctgatcaaatatgcgagcaacctggaatctggtgtgccggcgcgtttcagcggttctggcagcggcaccgacttcaccctgaacatccacccggtggaagaagaagataccgcgtattactattgccagcactcttgggaaatcccgcgtaccttcggtggcggcaccaaactggaaatcaaagaattcaacagcacgtaccgggttgtaagcgtcctcaccgtactgcaccaggactggctgaatggcaaggaatacaaatgcaagagtactgaggtgaagctggtggagtctggacctgagctggtgaagcctggagcttcaatgaagatatcctgcaaggcttctggttactcattcactggctacaccatgaactgggtgaagcagagtcatggaaagaaccttgagtggatgggacttattaatccttacaaaggtgttagtacctacaaccagaagttcaaggacaaggccacattaactgtagacaagtcatccagcacagcctacatggaactcctcagtctgacatctgaggactctgcagtctattactgtgcaagatcggggtactacggtgatagtgactggtacttcgatgtctggggcgcaggaacctcagtcactgtctcctcaactagtggtggtggtggttctggtggtggtggttctggtggtggtggttcttctagagacatccagatgacccagaccacatcctccctgtctgcctctctgggagacagagtcaccatcagttgcagggcaagtcaggacattagaaattatttaaactggtatcaacagaaaccagatggaactgttaaactcctgatctactacacatcaagattacactcaggagtcccatcaaagttcagtggcagtgggtctggaacagattattctctcaccattagcaacctggagcaagaggatattgccacttacttttgccaacagggtaatacgcttccgtggacgttcgctggaggcaccaaactggaactgaagcgcgctgtcgacttccagaatgcgctgctggttcgttacaccaagaaagtaccccaagtgtcaactccaactcctgtagaggtctcacatatgcaggtacagctacaggaatctggtccgggtctggtaaaaccgtctcagaccctgtctctgacctgtaccgtatctggtttctctctgtctgactatggtgttcattgggtacgtcagccgccaggtaaaggtctggaatgtctgggtgtaatatggggtggaggcacgaattataattcggctctcatgtccagacgtgtaacctcttccgacgatacctctaaaaatcagttctctctgaaactgtcttccgtagacaccgctgtatactattgtgctcgttcctattactattctatggactactggggtcagggcaccctggtaaccgtatcttccggtaccgaacaaaaactcatctcagaagaggatctgaatggggccgcacatcatcatcaccatcacgagcaa

SEQ ID NO: 4 - protein, 701 aa, Artificial Sequence
  CEA-scTsAb sequence
MGLEQVQLQQSGAELMKPGASVKISCKATGYTFSDYWIEWVKQRPGHGLEWIGEILPGSGRTDYNERFKGKATFTGDVSSNTAYMKLSSLTSEDSAVYYCATGTTPFGYWGQGTLVTVSATSTPSHNSHQVPSAGGPTANSGSRDIVLTQSPASLAVSLGQRATISCRASQSVSTSSYTYMHWYQQKPGQPPKLLIKYASNLESGVPARFSGSGSGTDFTLNIHPVEEEDTAYYYCQHSWEIPRTFGGGTKLEIKEFNSTYRVVSVLTVLHQDWLNGKEYKCKSTEVKLVESGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTSVTVSSTSGGGGSGGGGSGGGGSSRDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLELKRAVDFQNALLVRYTKKVPQVSTPTPVEVSHMQVQLQESGPGLVKPSQTLSLTCTVSGFSLSDYGVHWVRQPPGKGLECLGVIWGGGTNYNSALMSRRVTSSDDTSKNQFSLKLSSVDTAVYYCARSYYYSMDYWGQGTLVTVSSGTEQKLISEEDLNGAAHHHHHHEQ

SEQ ID NO: 5 - DNA, 18 bp, Artificial Sequence
  Synthetic fragment for making construct of CEA-scTsAb
tataccatgggtctcgag

SEQ ID NO: 6 - DNA, 59 bp, Artificial Sequence
  Synthetic fragment for making construct of CEA-scTsAb
tataccatgggtctcgagatgtacccgcgcggtaacactagtgaattcaacagcacgta

SEQ ID NO: 7 - DNA, 59 bp, Artificial Sequence
  Synthetic fragment for making construct of CEA-scTsAb
agccagtcctggtgcagtacggtgaggacgcttacaacccggtacgtgctgttgaattc

SEQ ID NO: 8 - DNA, 59 bp, Artificial Sequence
  Synthetic fragment for making construct of CEA-scTsAb
ctgcaccaggactggctgaatggcaaggaatacaaatgcaagagtacttctagaatgta

SEQ ID NO: 9 - DNA, 59 bp, Artificial Sequence
  Synthetic fragment for making construct of CEA-scTsAb
cgaaccagcagcgcattctggaagtcgacgttaccgcgcgggtacattctagaagtact

SEQ ID NO: 10 - DNA, 59 bp, Artificial Sequence
  Synthetic fragment for making construct of CEA-scTsAb
aatgcgctgctggttcgttacaccaagaaagtaccccaagtgtcaactccaactcctgt

SEQ ID NO: 11 - DNA, 59 bp, Artificial Sequence
  Synthetic fragment for making construct of CEA-scTsAb
gcggtaccgttaccgcgcgggtacatcatatgtgagacctctacaggagttggagttga

SEQ ID NO: 12 - DNA, 59 bp, Artificial Sequence
  Synthetic fragment for making construct of CEA-scTsAb
cgcggtaacggtaccgcgctggaagttgacgaaacctacgttccgaaagaatttaacgc

SEQ ID NO: 13 - DNA, 64 bp, Artificial Sequence
  Synthetic fragment for making construct of CEA-scTsAb
tcgctagccccatccgcgggatgtcagcgtggaaggtgaaggtttccgcgttaaattctttcgg

SEQ ID NO: 14 - DNA, 59 bp, Artificial Sequence
  Synthetic fragment for making construct of CEA-scTsAb
atcgagctcatgtacccgcgcggtaacgctagcgaacaaaaactcatctcagaagagga

SEQ ID NO: 15 - DNA, 59 bp, Artificial Sequence
  Synthetic fragment for making construct of CEA-scTsAb
tattgctcgtgatggtgatgatgatgtgcggccccattcagatcctcttctgagatgag

SEQ ID NO: 16 - DNA, 30 bp, Artificial Sequence
  Synthetic fragment for making construct of CEA-scTsAb
ctcgacggatccttattgctcgtgatggtg

SEQ ID NO: 17 - DNA, 22 bp, Artificial Sequence
  Synthetic fragment for making construct of CEA-scTsAb
taatacgactcactatagggga

SEQ ID NO: 18 - DNA, 19 bp, Artificial Sequence
  Synthetic fragment for making construct of CEA-scTsAb
gctagttattgctcagcgg

SEQ ID NO: 19 - DNA, 24 bp, Artificial Sequence
  Synthetic fragment for making construct of CEA-scTsAb
tcacatatgcaggtacagctacag

SEQ ID NO: 20 - DNA, 25 bp, Artificial Sequence
  Synthetic fragment for making construct of CEA-scTsAb
ttcgctagcggaagatacggtacca

SEQ ID NO: 21 - DNA, 25 bp, Artificial Sequence
  Synthetic fragment for making construct of CEA-scTsAb
aagagtactgaggtgaagctggtgg

SEQ ID NO: 22 - DNA, 27 bp, Artificial Sequence
  Synthetic fragment for making construct of CEA-scTsAb
gaagtcgacagcgcgcttcagttccag

SEQ ID NO: 23 - protein, 15 aa, Artificial Sequence
  Synthetic linker peptide linking VL and VH of anti-CEA monoclonal antibody
GGGGSGGGGSGGGGS

SEQ ID NO: 24 - DNA, 20 bp, Artificial Sequence
  Synthetic fragment for making construct of CEA-scTsAb
ttcctcgagcaggttcagct

SEQ ID NO: 25 - DNA, 58 bp, Artificial Sequence
  Synthetic fragment for making construct of CEA-scTsAb
tcgcgcccggtttcatcagttccgcaccgctctgctgcagctgaacctgctcgaggaa

SEQ ID NO: 26 - DNA, 58 bp, Artificial Sequence
  Synthetic fragment for making construct of CEA-scTsAb
actgatgaaaccgggcgcgagcgtgaaaatcagctgcaaagcgaccggctataccttc

SEQ ID NO: 27 - DNA, 44 bp, Artificial Sequence
  Synthetic fragment for making construct of CEA-scTsAb
cacccattcgatccaataatcgctgaaggtatagccggtcgctt

SEQ ID NO: 28 - DNA, 59 bp, Artificial Sequence
  Synthetic fragment for making construct of CEA-scTsAb
attattggatcgaatgggtgaaacagcgtccgggtcacggcctggaatggatcggtgaa

SEQ ID NO: 29 - DNA, 58 bp, Artificial Sequence
  Synthetic fragment for making construct of CEA-scTsAb
acgttcgttgtagtcggtacggccgctgcccggcaggatttcaccgatccattccagg

SEQ ID NO: 30 - DNA, 60 bp, Artificial Sequence
  Synthetic fragment for making construct of CEA-scTsAb
cgtaccgactacaacgaacgtttcaaaggcaaagcgaccttcaccggcgacgtttctagc

SEQ ID NO: 31 - DNA, 60 bp, Artificial Sequence
  Synthetic fragment for making construct of CEA-scTsAb
ttcgctggtcaggctagacagtttcatatacgcggtgttgctagaaacgtcgccggtgaa

SEQ ID NO: 32 - DNA, 59 bp, Artificial Sequence
  Synthetic fragment for making construct of CEA-scTsAb
tgtctagcctgaccagcgaagatagcgcggtgtattactgcgcgaccggcaccaccccg

SEQ ID NO: 33 - DNA, 60 bp, Artificial Sequence
  Synthetic fragment for making construct of CEA-scTsAb
gctcacggtcaccagggtgccctgaccccagtaaccgaacggggtggtgccggtcgcgca

SEQ ID NO: 34 - DNA, 59 bp, Artificial Sequence
  Synthetic fragment for making construct of CEA-scTsAb
gcaccctggtgaccgtgagcgcgactagtaccccgagccataacagccatcaggtgccg

SEQ ID NO: 35 - DNA, 59 bp, Artificial Sequence
  Synthetic fragment for making construct of CEA-scTsAb
gtctctagagccgctgttcgcggtcgggccgcccgcgctcggcacctgatggctgttat

SEQ ID NO: 36 - DNA, 58 bp, Artificial Sequence
  Synthetic fragment for making construct of CEA-scTsAb
cgaacagcggctctagagacatcgtgctgacccagagcccggcgagcctggcggtgtc

SEQ ID NO: 37 - DNA, 60 bp, Artificial Sequence
  Synthetic fragment for making construct of CEA-scTsAb
ctgggaagcacggcaggagatggtcgcacgctgacccagagacaccgccaggctcgccgg

SEQ ID NO: 38 - DNA, 59 bp, Artificial Sequence
  Synthetic fragment for making construct of CEA-scTsAb
tctcctgccgtgcttcccagtccgtttccacctcctcctacacctacatgcactggtat

SEQ ID NO: 39 - DNA, 56 bp, Artificial Sequence
  Synthetic fragment for making construct of CEA-scTsAb
gatcagcagtttcggcggctgacccggtttctgctgataccagtgcatgtaggtgt

SEQ ID NO: 40 - DNA, 59 bp, Artificial Sequence
  Synthetic fragment for making construct of CEA-scTsAb
agccgccgaaactgctgatcaaatatgcgagcaacctggaatctggtgtgccggcgcgt

SEQ ID NO: 41 - DNA, 59 bp, Artificial Sequence
  Synthetic fragment for making construct of CEA-scTsAb
gttcagggtgaagtcggtgccgctgccagaaccgctgaaacgcgccggcacaccagatt

SEQ ID NO: 42 - DNA, 59 bp, Artificial Sequence
  Synthetic fragment for making construct of CEA-scTsAb
gcaccgacttcaccctgaacatccacccggtggaagaagaagataccgcgtattactat

SEQ ID NO: 43 - DNA, 59 bp, Artificial Sequence
  Synthetic fragment for making construct of CEA-scTsAb
gccaccgaaggtacgcgggatttcccaagagtgctggcaatagtaatacgcggtatctt

SEQ ID NO: 44 - DNA, 50 bp, Artificial Sequence
  Synthetic fragment for making construct of CEA-scTsAb
tcccgcgtaccttcggtggcggcaccaaactggaaatcaaagaattcgcc

SEQ ID NO: 45 - DNA, 21 bp, Artificial Sequence
  Synthetic fragment for making construct of CEA-scTsAb
ggcgaattctttgatttccag

SEQ ID NO: 46 - DNA, 21 bp, Artificial Sequence
  Synthetic fragment for making construct of CEA-scTsAb
ggcgaattctttgatttccag

SEQ ID NO: 47 - DNA, 20 bp, Artificial Sequence
  Synthetic fragment for making construct of CEA-scTsAb
agccgccgaaactgctgatc

SEQ ID NO: 48 - DNA, 20 bp, Artificial Sequence
  Synthetic fragment for making construct of CEA-scTsAb
gatcagcagtttcggcggct

SEQ ID NO: 49 - DNA, 20 bp, Artificial Sequence
  Synthetic fragment for making construct of CEA-scTsAb
cgaacagcggctctagagac

SEQ ID NO: 50 - DNA, 20 bp, Artificial Sequence
  Synthetic fragment for making construct of CEA-scTsAb
gtctctagagccgctgttcg

SEQ ID NO: 51 - DNA, 20 bp, Artificial Sequence
  Synthetic fragment for making construct of CEA-scTsAb
gtaccgactacaacgaacgt

SEQ ID NO: 52 - DNA, 20 bp, Artificial Sequence
  Synthetic fragment for making construct of CEA-scTsAb
acgttcgttgtagtcggtac

SEQ ID NO: 53 - protein, 26 aa, Artificial Sequence
  FC linker
NSTYRVVSVLTVLHQDWLNGKEYKCK

SEQ ID NO: 54 - protein, 25 aa, Artificial Sequence
  HSA linker 1
FQNALLVRYTKKVPQVSTPTPVEVS

SEQ ID NO: 55 - protein, 24 aa, Artificial Sequence
  SHA linker 2
ALEVDETYVPKEFNAETFTFHADI

SEQ ID NO: 56 - protein, 11 aa, Artificial Sequence
  cmyc tag
EQKLISEEDLN

SEQ ID NO: 57 - protein, 6 aa, Artificial Sequence
  His tag
HHHHHH

SEQ ID NO: 58 - DNA, 437 bp, Artificial Sequence
  nucleotide sequence showing multiple cloning sites (fig 3)
tataccatgggtctcgagatgtacccgcgcggtaacactagtgaattcaacagcacgtaccgggttgtaagcgtcctcaccgtactgcaccaggactggctgaatggcaaggaatacaaatgcaagagtacttctagaatgtacccgcgcggtaacgtcgacttccagaatgcgctgctggttcgttacaccaagaaagtaccccaagtgtcaactccaactcctgtagaggtctcacatatgatgtacccgcgcggtaacggtaccgcgctggaagttgacgaaacctacgttccgaaagaatttaacgcggaaaccttcaccttccacgctgacatcccgcggatggggctagcgaacaaaaactcatctcagaagaggatctgaatggggccgcacatcatcatcaccatcacgagcaataaggatccgtcgag